(19) United States
(12) Patent Application Publication
(10) Pub. No.: US 2026/0167923 A1
(43) Pub. Date: Jun. 18, 2026

(54) IMMUNOGENIC ENGINEERED LIVE-ATTENUATED GRAM-NEGATIVE PATHOGEN VACCINES HAVING REDUCED ENDOTOXICITY

(71) Applicants: University of Maryland, Baltimore, Baltimore, MD (US); The Government of The United States, as represented by the Defense Health Agency, Fort Detrick, MD (US)

(72) Inventors: Robert K. Ernst, Silver Spring, MD (US); Matthew Sherman, Philadelphia, PA (US); Jane M. Wilhelm, Gwynn Oak, MD (US); Shoshana Barnoy, Rockville, MD (US); Malabi Venkatesan, Bethesda, MD (US)

(21) Appl. No.: 19/387,122

(22) Filed: Nov. 12, 2025

Related U.S. Application Data
(60) Provisional application No. 63/719,401, filed on Nov. 12, 2024.

(51) Int. Cl. C12N 1/20; C12N 9/18; C12N 15/74; C12R 1/00

(52) U.S. Cl. CPC C12N 1/20; C12N 9/18; C12N 15/74; C12N 2800/22; C12R 2001/00; C12Y 301/01077

(57) ABSTRACT

The present invention provides genetically modified Gram-negative microorganisms with reduced endotoxicity, wherein the Gram-negative microorganism is genetically modified to express a lipid A 1-phosphatase.

### CROSS-REFERENCE TO RELATED APPLICATIONS

This application claims the benefit of U.S. Provisional Appl. No. 63/719,401, filed Nov. 12, 2024, the contents of which are hereby incorporated by reference in their entirety.

### STATEMENT REGARDING FEDERALLY SPONSORED RESEARCH AND DEVELOPMENT

This invention was made with government support under W81XWH-19-2-0025 awarded by the Defense Health Agency, Medical Research and Development Branch. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 18,081 Byte XML file named “Sequence_Listing.xml,” created on Nov. 10, 2025.

### FIELD OF THE INVENTION

The invention relates to therapeutics and methods for treating or preventing Gram-negative infections, such as infections by Shigella, in particular using attenuated or live vaccines.

### BACKGROUND OF THE INVENTION

Shigella are Gram-negative bacteria known to cause diarrheal disease in humans through ingestion of contaminated food and water (Vos et al., The Lancet, (2016), 388(10053):1545-1602; Wang et al., The Lancet, (2016), 388(10053):1459-1544; Troeger et al., The Lancet Infectious Diseases, (2018), 18:1211-1228; Walker et al., Microorganisms, (2021), 9:1382). Of the four pathogenic Shigella species (S. sonnei, S. flexneri, S. boydii, and S. dysenteriae), S. sonnei (Ss) and S. flexneri (Sf) cause the majority of disease in industrialized and developing countries, respectively (Pasetti et al, Mucosal Vaccines, (2020), 515-536). Shigellosis (Shigella-induced diarrhea) affects all age groups but particularly plagues young children as uncontrolled inflammation and severe dehydration can lead to growth abnormalities, seizure, and even death (Wang et al., The Lancet, (2016), 388(10053):1459-1544; Liu et al., The Lancet, (2016), 388: 1291-1301; Khalil et al., The Lancet Infectious Diseases, (2018), 18:1229-1240; Williams et al., Paediatrics and International Child Health, (2018), 38: S50-S65; Anderson et al., The Lancet Global Health, (2019), 7: e321-e330). The clinical severity and emergence of antibiotic resistance have prompted the development of multiple Shigella vaccine candidates that are currently in preclinical and clinical phases (Walker et al., Microorganisms, (2021), 9:138; Schroeder et al., Clin Microbiol Rev, (2008), 21:134-56; Kotloff et al., The Lancet, (2018), 391:801-812; Shad et al., Archives of Microbiology, (2021), 203:45-58; Network, C. H. A. Increase in Extensively Drug-Resistant Shigellosis in the United States, (2023), Feb. 24, 2023; Barry et al., Nature Reviews Gastroenterology & Hepatology, (2013), 10: 245-255). However, to date, there is no FDA-licensed Shigella vaccine; Protection against shigellosis is serotype-specific (Walker et al., Microorganisms, (2021), 9:1382; Pasetti et al, Mucosal Vaccines, (2020), 515-536; Barry et al., Nature Reviews Gastroenterology & Hepatology, (2013), 10: 245-255; Levine et al., Nature Reviews Microbiology, (2007), 5: 540-553), implicating the O-antigen component of lipopolysaccharide (LPS) as being the critical antigen for vaccine development. Vaccine strategies to prevent shigellosis have included glycoconjugate vaccines where the O-antigen is conjugated to carrier adjuvants (Walker et al., Microorganisms, (2021), 9:1382; Pasetti et al, Mucosal Vaccines, (2020), 515-536; Barry et al., Nature Reviews Gastroenterology & Hepatology, (2013), 10: 245-255; Levine et al., Nature Reviews Microbiology, (2007), 5: 540-553; Kaminski et al., Expert Rev Vaccines, (2009), 8:1693-704), an oligomeric complex comprised of LPS and the invasion plasmid antigens (Ipa) called Invaplex (Turbyfill et al., mSphere, (2018), 3(2); Duplessis et al., Vaccine, (2023), 41: 6261-627; Turbyfill et al., mSphere, (2023), 8:e0007323), and outer-membrane vesicles (OMVs) produced via the generalized modules for membrane antigens (GMMA) method (Berlanda Scorza, F., PLoS One, (2012), 7: e35616; Rossi et al., Journal of Biological Chemistry, (2014), 289:24922-24935; Gerke et al., PLOS ONE, (2015), 10: e0134478; Conti et al., NPJ Vaccines, (2024), 9: 56). Another longstanding strategy has been the development of live-attenuated Shigella strains where the O-antigen remains in its native context on the outer membrane (Walker et al., Microorganisms, (2021), 9:1382; Pasetti et al, Mucosal Vaccines, (2020), 515-536; Barry et al., Nature Reviews Gastroenterology & Hepatology, (2013), 10: 245-255; Levine et al., Nature Reviews Microbiology, (2007), 5: 540-553; Venkatesan et al., Expert Rev Vaccines, (2006), 5:669-86). Current live-attenuated vaccine candidates focus primarily on S. sonnei and S. flexneri serotype 2a, which are epidemiologically significant Shigella strains and whose O-antigen structures are well characterized (Levine et al., Nature Reviews Microbiology, (2007), 5: 540-553; Livio et al., Clinical Infectious Diseases, (2014), 59:933-941).

A key advantage of live-attenuated Shigella vaccines, which generally contain genetic mutations or deletions in virulence-associated genes, is that they mimic a natural Shigella infection and, therefore, generate an immune response that protects against future infection (Pasetti et al, Mucosal Vaccines, (2020), 515-536; Barry et al., Nature Reviews Gastroenterology & Hepatology, (2013), 10: 245-255). While these vaccines induce protective immune responses against virulent challenge in volunteer studies, significant issues including adverse reactogenicity have slowed the progress toward a universally accepted safe and effective Shigella vaccine (Coster et al., Infection and Immunity, (1999), 67:3437-3443; Walker et al., Microorganisms, (2021), 9:1382; Pasetti et al, Mucosal Vaccines, (2020), 515-536; Barry et al., Nature Reviews Gastroenterology & Hepatology, (2013), 10: 245-255).

LPS is a glycolipid present on the outer membrane of Gram-negative bacteria and is composed of three regions: the O-antigen, core oligosaccharide, and lipid A membrane anchor (Miller et al., Nature Reviews Microbiology, (2005), 3:36-46; Chandler et al., F1000Research, (2017), 6:1334). Innate immune cells can recognize the lipid A region of LPS and initiate cytokine production to alert the immune system to the presence of a bacterial invader (Zamyatina et al., Front Immunol, (2020), 11:585146). This occurs through a series of accessory proteins that guide the binding of lipid A to the TLR4/MD-2 receptor on the surface of innate immune cells to drive downstream signaling, such as the NF-κB pathway, and induce pro-inflammatory cytokine production (Scott et al., Biochimica et Biophysica Acta (BBA)—Molecular and Cell Biology of Lipids, (2017), 1862:1439-1450; Park et al., Nature, (2009), 458:1191-1195). The TLR4/MD-2 response is primarily driven by the structural features of lipid A, which vary across Gram-negative bacteria (Zamyatina et al., Front Immunol, (2020), 11:585146; Scott et al., Biochimica et Biophysica Acta (BBA)—Molecular and Cell Biology of Lipids, (2017), 1862:1439-1450; Raetz et al., Annual Review of Biochemistry, (2007), 76:295-329; Simpson et al., Nature Reviews Microbiology, (2019), 17:403-416). Shigella synthesizes a prototypical lipid A structure comprised of six acyl chains (hexa-acylated) and two terminal phosphates (bis-phosphorylated) (Zamyatina et al., Front Immunol, (2020), 11:585146). This structure is a potent stimulator of TLR4/MD-2, and the ensuing pro-inflammatory cytokine production likely contributes to the febrile symptoms observed upon oral ingestion of Shigella for vaccination (Rossi et al., Journal of Biological Chemistry, (2014), 289:24922-24935).

What is needed are new and improved agents to treat or prevent Shigella infection, including Shigella vaccines that are both highly immunogenic but having reduced reactogenicity. This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

In this disclosure, the inventors have modified live-attenuated Shigella strains with the aim of reducing their endotoxicity. The present inventors have modified WRSs2 (S. sonnei strain) and WRSf2G12 (S. flexneri 2a strain), developed by Walter Reed Army Institute of Research (WRAIR), which are principally attenuated by deletion of virG (also known as icsA) thereby prohibiting intercellular spread but also contain deletions in genes encoding enterotoxins (Barzu et al., Infect Immun, (1996), 64:1190-6; Hartman et al., Infect Immun, (1998), 66:4572-6; Collins et al., Comp Med, (2008), 58:88-94; Barnoy et al., Vaccine, (2010), 28:1642-1654; Barnoy et al., Vaccine, (2011), 29:6371-6378; Bedford et al., Gut Microbes, (2011), 2:244-251; Ranallo et al., Vaccine, (2012), 30:5159-5171; Jeong et al., Vaccine, (2013), 31:4039-4046; Ranallo et al., Vaccine, (2014), 32:1754-1760). WRSs2 and WRSf2G12 are second-generation vaccines whose first-generation counterparts (WRSS1 and SC602, respectively) were highly immunogenic in adults and children during clinical trials, but substantial reactogenicity was observed at the moderate to high doses required to confer protective immunity (Coster et al., Infection and Immunity, (1999), 67:3437-3443; Kotloff et al., Infect Immun, (2002), 70:2016-21; Katz et al., Infection and Immunity, (2004), 72:923-930; Orr et al., Infect Immun, (2005), 73:8027-32; Rahman et al., Vaccine, (2011), 29:1347-1354; Pitisuttithum et al., Clinical and Vaccine Immunology, (2016), 23:564-575; Raqib et al., Human Vaccines & Immunotherapeutics, (2019), 15:1326-1337). In a phase I clinical trial, WRSs2 was generally well tolerated but still not at a satisfactory level where it can be used as a safe vaccine (Frenck et al., Vaccine, (2018), 36:4880-4889). To improve the safety of WRSs2 and WRSf2G12 the present inventors targeted the highly immunostimulatory LPS molecule present on the bacterial membrane, which is thought to be a major contributor to these adverse effects.

This disclosure describes bacterial enzymatic combinatorial chemistry (BECC) to detoxify WRSs2 and WRSf2G12, as well as their respective wild-type strains (Ernst et al., Hajjar, Immunotherapeutic potential of modified lipooligosaccharides/lipid a. United States). Lipid A modifying enzymes LpxE (1-position phosphatase from Francisella) and PagL (3-position deacylase from Salmonella) were ectopically expressed in the Shigella backgrounds. Targeted lipid A dephosphorylation (LpxE), deacylation (PagL), or execution of both modifications (Dual) was confirmed using MALDI-TOF MS. Lipid A dephosphorylation, rather than deacylation, effectively diminished LPS-induced pro-inflammatory immune signaling. Furthermore, deacylation combined with dephosphorylation did not further reduce LPS-mediated signaling. Due to this, only dephosphorylated lipid A mutants (LpxE-modified) were generated in WRSs2 and WRSf2G12, generating WRSs2E and WRSf2G12E. These strains had reduced LPS-mediated pro-inflammatory cytokine production in vitro and severely blunted endotoxemia in vivo, yet they remained as capable as their parental strains to invade epithelia and generate immunogenicity against their O-antigen. Two live-attenuated Shigella vaccine candidates (WRSs2E and WRSf2G12E), altered only in their lipid A region, are characterized which are greatly detoxified without any consequence to phenotypic traits suggesting that such live-attenuated strains are a promising approach to develop a safe and effective Shigella vaccine.

In one aspect, the invention provides a genetically modified Gram-negative microorganism with reduced endotoxicity, wherein the microorganism is genetically modified to express a lipid A 1-phosphatase. In some embodiments, the lipid A 1-phosphatase is LpxE. In some embodiments, the lipid A 1-phosphatase is from a Gram-negative bacteria. In some embodiments, the LpxE is from Francisella novicida.

In some embodiments, the Gram-negative microorganism is a pathogen. In some embodiments, the pathogen is selected from Shigella, Escherichia coli, Pseudomonas aeruginosa, Salmonella, Vibrio and Yersinia species.

In some embodiments, the lipid A 1-phosphatase is codon optimized for expression in the Gram-negative microorganism, such as Shigella.

In some embodiments, the LpxE has an animo acid sequence comprising SEQ ID NO:1. In some embodiments, the LpxE is encoded by a nucleic acid sequence comprising SEQ ID NO:2.

In some embodiments, the Gram-negative microorganism, such as Shigella is genetically modified to express a 3-O deacylase. In some embodiments, the 3-O deacylase is PagL. In some embodiments, the PagL is from Salmonella enterica. In some embodiments, the PagL is codon optimized for expression in the Gram-negative microorganism, such as Shigella. In some embodiments, the PagL has an animo acid sequence comprising SEQ ID NO:3. In some embodiments, the PagL is encoded by a nucleic acid sequence comprising SEQ ID NO:4.

In some embodiments, the lipid A 1-phosphatase is chromosomally integrated. In some embodiments, the lipid A 1-phosphatase is expressed from a plasmid. In some embodiments, the lipid A 1-phosphatase is regulated by an inducible promoter. In some embodiments, the inducible promoter is an osmotically controlled E. coli ompC promoter (P_{ompC}).

In some embodiments, the lipid A 1-phosphatase and/or the 3-O deacylase are chromosomally integrated at the attTn7 site in the Gram-negative microorganism, such as Shigella.

In some embodiments, the 3-O deacylase and/or lipid A 1-phosphatase are chromosomally integrated. In some embodiments, the 3-O deacylase and/or lipid A 1-phosphatase is expressed from a plasmid. In some embodiments, the 3-O deacylase and/or lipid A 1-phosphatase is regulated by an inducible promoter. In some embodiments, the inducible promoter is an osmotically controlled E. coli ompC promoter (P_{ompC}).

In some embodiments, the LpxE is located downstream of an osmotically controlled E. coli ompC promoter (P_{ompC}) and has a nucleotide sequence comprising SEQ ID NO:5.

In some embodiments, the lipid A 1-phosphatase and the 3-O deacylase are expressed from a single cassette.

In some embodiments, the Gram-negative microorganism, such as Shigella is attenuated. In some embodiments, the Gram-negative microorganism, such as Shigella has more than one attenuating mutations. In some embodiments, the Gram-negative microorganism, such as Shigella, has a deletion in one or more genes selected from virG, senA, senB, setAB, iuc and combinations thereof. In some embodiments, the Gram-negative microorganism, such as Shigella has a deletion in virG. In some embodiments, the Gram-negative microorganism, such as Shigella has a deletion in virG, senA, and senB. In some embodiments, the Gram-negative microorganism, such as Shigella has a deletion in virG and iuc. In some embodiments, the Gram-negative microorganism, such as Shigella has a deletion in virG, senA, senB, and setAB.

In some embodiments, the Shigella is selected from S. sonnei and S. flexneri. In some embodiments, the S. sonnei is S. sonnei Moseley. In some embodiments, the S. flexneri is S. flexneri 2a 2457T.

In some embodiments, the S. sonnei strain WRSS1 is genetically modified.

In some embodiments, the S. sonnei strain WRSs2 is genetically modified.

In some embodiments, the S. flexneri 2a strain SC602 is genetically modified.

In some embodiments, the S. flexneri 2a strain WRSf2G12 is genetically modified.

In some embodiments, the lipid A molecules of the Gram-negative microorganism, such as Shigella lack a phosphate at the 1-position. In some embodiments, the lipid A molecules of the Gram-negative microorganism, such as Shigella lack an acyl chain at the 3-position.

In another aspect, the invention provides a pharmaceutical composition comprising the genetically modified Gram-negative microorganism, such as Shigella as described herein. In some embodiments, the pharmaceutical composition is formulated for oral administration. In some embodiments, the pharmaceutical composition is formulated for intranasal administration.

In another aspect, the invention provides a method of inducing an immune response in a subject, comprising administering to the subject an immunologically-effective amount of the pharmaceutical compositions herein.

In some embodiments, the pharmaceutical composition is orally administered. In some embodiments, the pharmaceutical composition is intranasally administered. In some embodiments, the method comprises administering a combination of genetically modified Gram-negative microorganisms, such as Shigella microorganisms. In some embodiments, the combination comprises a plurality of Shigella strains. In some embodiments, the combination comprises S. sonnei and S. flexneri.

In some embodiments, the subject is first administered the immunologically-effective amount of the pharmaceutical composition as a prime and is subsequently administered an immunologically-effective amount of the pharmaceutical composition as a boost.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. Lipid A modifications generated by BECC constructs. (A) The WT lipid A structure of Shigella along with the (B) LpxE- (C) PagL- and (D) Dual-modified resultant structures, lacking a phosphate, 3OH C14 acyl chain, or both, respectively. The expected m/z for the [M-H]⁻ ions observed in MALDI-TOF spectra are displayed for each structure.

FIG. 2. MALDI-TOF MS analysis of lipid A related peaks from BECC-modified Shigella. Representative MALDI-TOF MS spectra for (A) S. sonnei and (B) S. flexneri 2a strains chromosomally expressing lpxE or Dual. Spectral peaks represent [M-H]⁻ ions. Colored peaks correspond to the expected structures detailed in FIG. 1. Arrows depict the loss of a phosphate (HPO₃) or acyl chain (3OH C14) from the indicated lipid A species.

FIG. 3. Invasion of epithelial cells by Shigella and corresponding CXCL8 production. Invasion of HT29 cells after 4 hours of infection (MOI of 10) with (A) S. sonnei and (B) S. flexneri 2a strains. CXCL8 production in the cell supernatant after the 4-hour infection with (C) S. sonnei and (D) S. flexneri 2a strains. Statistical significance determined by ordinary one-way ANOVA. * and *** represent p-values of ≤0.05 and ≤0.001, respectively.

FIG. 4. Endotoxicity of BECC-modified LPS from WRSs2 and WRSf2G12 in vitro and in vivo. HEK 293 NF-κB reporter cells (HEK-Blue) overexpressing the human (hTLR4) or mouse (mTLR4) orthologs of TLR4/MD-2/CD-14 were stimulated across 10-fold dilutions, in duplicate, of Kdo standardized LPS from (A) WRSf2G12 and WRSf2G12E or (B) WRSs2 and WRSs2E for 18 hours at 37° C. with 5% CO₂. (C) Human PBMCs from four independent donors, plotted as lines for each donor, were stimulated with 1 μg/mL of Kdo normalized LPS for 48 hours at 37° C. with 5% CO₂ and cytokine levels in the supernatant quantified by multiplex analysis. The LPS used was purified from the indicated strain below the x-axis. (D) Survival curves for mice (n=5) receiving a Kdo normalized dose of LPS intraperitoneally, representative of 15 mg/kg, using purified LPS from the indicated strains.

FIG. 5. Antibody titers from a Shigella murine vaccine study. Serum IgG and IgA geometric mean titers against (A) Moseley or (B) 2457T LPS for mice (n=15) vaccinated intranasally with 10⁶ CFU at day 0, 14, and 28 as indicated by the arrows below the x-axis. (C) Serum IgG2a and IgG1 titers at day 56 against serotype-specific LPS. Statistical significance was determined by 2way ANOVA. * and **** represent p-values of ≤0.05 and ≤0.0001, respectively.

FIG. 6. MALDI-TOF MS analysis of Shigella strains ectopically expressing lipid A modifying enzymes from the pSEC10M expression plasmid. Representative MALDI-TOF MS spectra from strains of (A) S. sonnei Moseley, (B) S. flexneri 2a 2457T, (C) WRSS1, and (D) SC602. Spectral peaks represent [M-H]⁻ ions. Colored peaks correspond to the expected structures detailed in FIG. 1. Arrows depict the loss of a phosphate (HPO₃) or acyl chain (3OH C14) from the indicated lipid A species.

FIG. 7. Stimulation of NF-κB reporter cells with Kdo normalized LPS from Shigella strains expressing lipid A modifying enzymes from the pSEC10M expression plasmid. HEK-Blue cells overexpressing the human or mouse orthologs of TLR4/MD-2/CD-14 (denoted hTLR4 or mTLR4, respectively) were stimulated across 10-fold dilutions, in duplicate, of Kdo standardized LPS for 18 hours at 37° C. with 5% CO₂. LPS was purified from (A) S. sonnei Moseley or (B) S. flexneri 2a 2457T.

FIG. 8. MALDI-TOF MS/MS analysis of WT and BECC-modified lipid A from Shigella. Lipid A variants were processed via the FLATⁿ method and analyzed via MS/MS on a Bruker timsTOF. (A) Precursor ions, m/z 1797, 1717, and 1490, specific to WT, LpxE-, and Dual-modified lipid A, respectively, were selected for fragmentation. (B) Resultant fragmentation of the precursor ions (circled in red). (C) Lipid A structures showing fragment ion breakages correlated to the assignments above the spectral peaks in (B).

FIG. 9. SDS-PAGE and Pro-Q Emerald 300 analysis of LPS.

FIG. 10. Growth curves for Shigella strains used in this study. Growth over time for 10⁵ CFU inoculums in 96-well plates of WT and vaccine strains of (A) S. sonnei and (B) S. flexneri 2a and their BECC variants. Plotted values are the mean of triplicate readings. Error bars are omitted for clarity.

FIG. 11. Endotoxicity of BECC-modified LPS from Moseley and 2457T in vitro and in vivo. HEK 293 NF-κB reporter cells (HEK-Blue) overexpressing the human (hTLR4) or mouse (mTLR4) orthologs of TLR4/MD-2/CD-14 were stimulated across 10-fold dilutions, in duplicate, of Kdo standardized LPS from (A) 2457T or (B) Moseley for 18 hours at 37° C. with 5% CO₂. (C) Human PBMCs from four independent donors, plotted as lines for each donor, were stimulated with 1 μg/mL of Kdo normalized LPS for 48 hours at 37° C. with 5% CO₂ and cytokine levels in the supernatant quantified by multiplex analysis. The LPS used was purified from the indicated strain below the x-axis. (D) Repeated study with a different set of PBMCs using 1 μg/mL of Kdo normalized LPS from Moseley and a shorter stimulation of 24 hours instead. (E) Survival curves for mice (n=5) receiving a Kdo normalized dose of LPS intraperitoneally, representative of 15 mg/kg, using purified LPS from the indicated strains.

FIG. 12. Stimulation of macrophage NF-κB reporter cells with Kdo normalized LPS. Macrophage NF-κB reporter cells endogenously expressing the human (THP-1-Dual) or mouse (RAW-Blue) orthologs of TLR4/MD-2/CD-14 were stimulated across 10-fold dilutions, in duplicate, of Kdo standardized LPS for 18 hours at 37° C. with 5% CO₂. The LPS used was purified from the indicated strains in each panel.

FIG. 13. Optimization of the Shigella murine vaccination model. (A) Various immunization routes were tested in a prime-boost-boost model using either WRSs2 or purified LPS from WT Moseley. (B) Serum IgG and IgA antibody titers over time against WT Moseley LPS. Higher dose groups are indicated by dashed lines. Arrows indicate the day of administration for the vaccine doses. Only the geometric means are plotted. The errors bars are omitted for clarity. Abbreviations include i.n. for intranasal and o.g. for oral gastric.

FIG. 14. Antibody titers from a second independent Shigella murine vaccine study. Serum IgG and IgA geometric mean titers against (A) Moseley or (B) 2457T LPS for mice (n=15) vaccinated intranasally with 10⁶ CFU at day 0, 14, and 28 as indicated by the arrows below the x-axis. (C) Serum IgG2a and IgG1 titers at day 56 against serotype-specific LPS. Arrows in indicate administration of vaccine doses. Statistical significance determined by 2way ANOVA. **, **** represent p-values of ≤0.01, and ≤0.0001, respectively.

FIG. 15. Schematic of the current and future engineering of Shigella, resulting lipid A modification and effects.

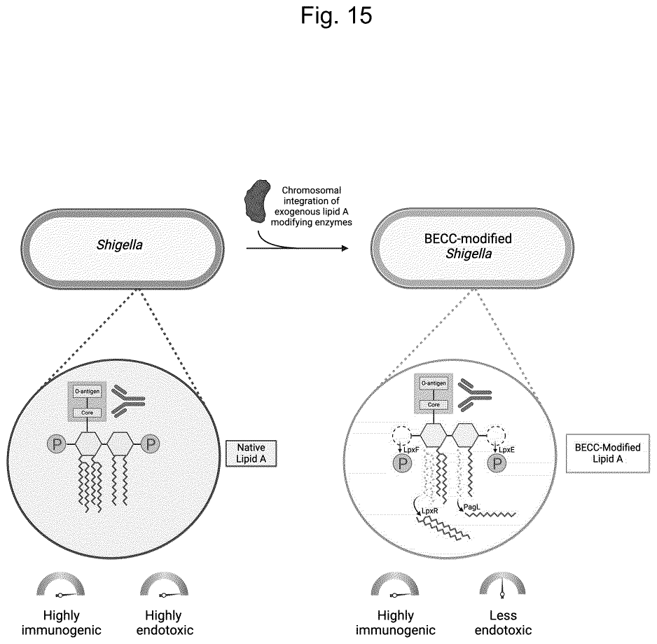

### DETAILED DESCRIPTION OF THE INVENTION

Shigella spp. infection contributes significantly to the global disease burden, primarily affecting young children in developing countries. Currently, there are no FDA-approved vaccines against Shigella, and the prevalence of antibiotic resistance is increasing, making therapeutic options limited. Live-attenuated vaccine strains WRSs2 (S. sonnei) and WRSf2G12 (S. flexneri 2a) are highly immunogenic, making them promising vaccine candidates, but possess an inflammatory lipid A structure on their lipopolysaccharide (LPS; also known as endotoxin). Here, we utilized bacterial enzymatic combinatorial chemistry (BECC) to ectopically express lipid A modifying enzymes in WRSs2 and WRSf2G12, as well as their respective wild-type strains, generating targeted lipid A modifications across the Shigella backgrounds. Dephosphorylation of lipid A, rather than deacylation, reduced LPS-induced TLR4 signaling in vitro and dampened endotoxic effects in vivo. These BECC-modified vaccine strains retained the phenotypic traits of their parental strains, such as invasion of epithelial cells and immunogenicity in mice without adverse endotoxicity. Overall, our observations suggest that BECC-engineered live attenuated vaccines are a promising approach to safe and effective Shigella vaccines.

Reference will now be made in detail to the presently preferred embodiments of the invention which, together with the drawings and the following examples, serve to explain the principles of the invention. These embodiments are described in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual (Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means plus or minus 10% of the numerical value of the number with which it is being used.

“Lipid A” is considered to be the hydrophobic anchor of LPS and is a glucosamine-based lipid that makes up the outer monolayer of the outer membrane of Gram-negative bacteria. See, e.g., Raetz, et al., Discovery of new biosynthetic pathways: the lipid A story, J. Lipid Res. 2009, 50(Suppl): S103-S108. Unless otherwise indicated, “lipid A” in the present application means Shigella lipid A.

“Lipopolysaccharides” (LPS) are outer membrane components of gram-negative bacteria. LPS include lipid A and can induce an innate immune response in humans and other subjects. See, e.g., A. Farhana, et al., Biochemistry, Lipopolysaccharide (Treasure Island, FL: StatPearls Publishing, 2023).

“Lipooligosaccharide” (LOS) is a glycolipid found in the outer membranes of some Gram-negative bacteria. LOS includes lipid A and has a relative paucity of glycosylation in comparison to lipopolysaccharide of enteric Gram-negative bacterial species. See, e.g., J. A. Duncan, et al., Gonococcal and other neisserial infections, in Tropical Infectious Diseases (R. L. Guerrant, D. H. Walker and P. F. Weller, eds., 3rd ed., Elsevier Inc., 2011).

“Monophosphoryl lipid A” (MPL) is a non-toxic TLR4 ligand, derived from Salmonella Minnesota R595 (Re) lipopolysaccharide (LPS) by chemical modification. See, e.g., S. Schulke, et al., Monophosphoryl lipid A as an adjuvant for immune therapy? A detailed in vitro comparison to LPS, Clin. Transl. Allergy 2014, 4(Suppl 2): O21.

### Genetically Modified Gram-Negative Microorganism

In one aspect, the invention provides a genetically modified Gram-negative microorganism with reduced endotoxicity, wherein the Gram-negative microorganism is genetically modified to express a lipid A 1-phosphatase. In some embodiments, the lipid A 1-phosphatase is LpxE. In some embodiments, the LpxE is from Francisella novicida. In some embodiments, the lipid A 1-phosphatase is codon optimized for expression in the Gram-negative microorganism. In some embodiments, the LpxE has an animo acid sequence comprising SEQ ID NO:1. In some embodiments, the LpxE is encoded by a nucleic acid sequence comprising SEQ ID NO:2.

In some embodiments, the Gram-negative microorganism is genetically modified to express a 3-O deacylase. In some embodiments, the 3-O deacylase is PagL. In some embodiments, the PagL is from Salmonella enterica. In some embodiments, the PagL is codon optimized for expression in Shigella. In some embodiments, the PagL has an animo acid sequence comprising SEQ ID NO:3. In some embodiments, the PagL is encoded by a nucleic acid sequence comprising SEQ ID NO:4.

| | |
|---|---|
| SEQ ID NO: 1 | MLKQTLQTNF QGFKDIFKKP KLHNHKLPRY |
| | LQLKYTFIPL LILVIFAYYN LDTPVENYIK |
| | HSMPNIVGVI FGKITDVGKA EYILIICGVI |
| | VLARLFTDSQ KLSANTRAMF DKVSAYAGFI |
| | LATVAISGIL GQILKMIIGR ARPKFFLEYG |
| | SHYFQHFHAP GYDFASMPSG HSITVGAMFI |
| | AFFYIFPKLR YFWYLLIVVF AGSRIMVGSH |
| | YPSDVIFGVA FGCYCTAYIY YWMRNREII |
| SEQ ID NO: 2 | ATGCTCAAACAGACC TTACAAACCA |
| | ACTTTCAAGG TTTTAAAGAT ATTTTTAAAA |
| | AACCGAAACT GCACAATCAT AAATTGCCCA |
| | GATACCTGCA GTTGAAATAT ACGTTTATTC |
| | CGTTATTAAT TTTGGTAATT TTTGCATACT |
| | ATAACTTAGA TACCCCGGTT GAGAACTATA |
| | TCAAGCATTC TATGCCGAAT ATTGTTGGTG |
| | TAATTTTTGG TAAAATTACG GATGTTGGTA |
| | AGGCCGAGTA TATTTTGATT ATTTGCGGTG |
| | TGATTGTGTT AGCGCGTTTA TTTACCGATA |
| | GCCAAAAATT ATCTGCTAAT ACGAGAGCTA |
| | TGTTTGACAA GGTGTCGGCA TATGCGGGTT |
| | TTATCTTAGC AACGGTAGCT ATTAGCGGTA |
| | TTTTGGGACA AATTCTCAAG ATGATTATTG |
| | GTAGAGCGCG TCCTAAGTTT TTCTTGGAAT |
| | ATGGTTCGCA TTATTTCCAA CATTTTCATG |
| | CACCCGGATA TGATTTTGCA AGCATGCCGT |
| | CCGGGCATTC CATCACCGTT GGAGCAATGT |
| | TTATTGCATT TTTTTATATT TTCCCTAAGC |
| | TGAGATATTT TTGGTATTTG CTGATTGTGG |
| | TATTTGCTGG GAGCAGAATT ATGGTTGGTT |
| | CACATTATCC GAGCGATGTA ATTTTTGGCG |
| | TTGCTTTTGG TTGCTACTGT ACCGCATATA |
| | TCTACTATTG GATGAGAAAT AGAGAGATTA |
| | TTTAA |
| SEQ ID NO: 3 | MKRIFIYLLL PCAFACSAND NVFFGKGNKH |
| | QISFAAGESI RRGGVEHLYT AFLTYSEPSD |
| | FFFLQARNNL ELGGFKAKGS DDCSKHSGSV |
| | PCNKYNQGVL GISKDVALVH FAGIYTGIGL |
| | GAYIKSKSRD DMRVNSAFTF GEKAFLGWNF |
| | GAFSTEAYIR HFSNGSLTDK NSGHNFVGAS |
| | ISYNF |
| SEQ ID NO: 4 | ATGAAGAGAA TATTTATATA TCTATTATTA |
| | CCTTGTGCAT TCGCATGTTC TGCTAATGAT |
| | AATGTTTTTT TTGGCAAGGG CAACAAGCAT |
| | CAGATCTCTT TTGCTGCGGG AGAAAGTATA |
| | AGAAGAGGAG GGGTTGAGCA CTTATATACG |
| | GCTTTTCTGA CATACAGTGA ACCCAGCGAT |
| | TTTTTCTTTT TACAGGCAAG AAATAATCTG |
| | GAGTTAGGAG GATTTAAGGC TAAGGGTAGC |
| | GATGATTGCA GTAAACATTC TGGCAGCGTT |
| | CCCTGTAATA AATATAACCA GGGCGTATTG |
| | GGTATCTCGA AGGATGTGGC GCTGGTTCAT |
| | TTCGCTGGTA TCTATACCGG TATTGGTCTG |
| | GGGGCTTATA TAAAATCTAA GTCGCGAGAT |
| | GATATGCGTG TCAATTCTGC ATTTACCTTT |
| | GGAGAAAAAG CGTTTCTTGG CTGGAACTTT |
| | GGGGCTTTTT CTACAGAAGC TTATATCCGG |
| | CATTTCTCGA ATGGATCACT TACGGATAAA |
| | AATTCAGGGC ATAATTTTGT AGGTGCTTCA |
| | ATTAGTTATA ATTTCTGA |
| SEQ ID NO: 5 | GAATTCTGTG GTAGCACAGA ATAATGAAAA |
| | GTGTGTAAAG AAGGGTAAAA AAAACCGAAT |
| | GCGAGGCATC CGGTTGAAAT AGGGGTAAAC |
| | AGACATTCAG AAATGAATGA CGGTAATAAA |
| | TAAAGTTAAT GATGATAGCG GGAGTTATTC |
| | TAGTTGCGAG TGAAGGTTTT GTTTTGACAT |
| | TCAGTGCTGT CAAATACTTA AGAATAAGTT |
| | ATTGATTTTA ACCTTGAATT ATTATTGCTT |
| | GATGTTAGGT GCTTATTTCG CCATTCCGCA |
| | ATAATCTTAA AAAGTTCCCT TGCATTTACA |
| | TTTTGAAACA TCTATAGCGA TAAATGAAAC |
| | ATCTTAAAAG TTTTAGTATC ATATTCGTGT |
| | TGGATTATTC TGCATTTTTG GGGAGAATGG |
| | ACTTGCCGAC TGATTAATGA GGGTTAATCA |
| | GTATGCAGTG GCATAAAAAA GCAAATAAAG |
| | GCATATAACA GATCGATCTT AAACATCCAC |
| | AGGAGGATGG GATCCATGCT CAAACAGACC |
| | TTACAAACCA ACTTTCAAGG TTTTAAAGAT |
| | ATTTTTAAAA AACCGAAACT GCACAATCAT |
| | AAATTGCCCA GATACCTGCA GTTGAAATAT |
| | ACGTTTATTC CGTTATTAAT TTTGGTAATT |
| | TTTGCATACT ATAACTTAGA TACCCCGGTT |
| | GAGAACTATA TCAAGCATTC TATGCCGAAT |
| | ATTGTTGGTG TAATTTTTGG TAAAATTACG |
| | GATGTTGGTA AGGCCGAGTA TATTTTGATT |
| | ATTTGCGGTG TGATTGTGTT AGCGCGTTTA |
| | TTTACCGATA GCCAAAAATT ATCTGCTAAT |
| | ACGAGAGCTA TGTTTGACAA GGTGTCGGCA |
| | TATGCGGGTT TTATCTTAGC AACGGTAGCT |
| | ATTAGCGGTA TTTTGGGACA AATTCTCAAG |
| | ATGATTATTG GTAGAGCGCG TCCTAAGTTT |
| | TTCTTGGAAT ATGGTTCGCA TTATTTCCAA |
| | CATTTTCATG CACCCGGATA TGATTTTGCA |
| | AGCATGCCGT CCGGGCATTC CATCACCGTT |
| | GGAGCAATGT TTATTGCATT TTTTTATATT |
| | TTCCCTAAGC TGAGATATTT TTGGTATTTG |
| | CTGATTGTGG TATTTGCTGG GAGCAGAATT |
| | ATGGTTGGTT CACATTATCC GAGCGATGTA |
| | ATTTTTGGCG TTGCTTTTGG TTGCTACTGT |
| | ACCGCATATA TCTACTATTG GATGAGAAAT |
| | AGAGAGATTA TTTAA |

In some embodiments, the Gram-negative microorganism is a pathogen. In some embodiments, the pathogen is selected from Shigella, Escherichia coli, Pseudomonas aeruginosa, Salmonella, Vibrio and Yersinia species.

Strains expressing functional variants or fragments (e.g., N-terminal or C-terminal deletions) of LpxE and/or PagL are also contemplated, e.g., amino acid sequences having at least 90%, 91%, 92%, 93%, 94%, 95%, 96%, 97%, 98%, 99% or greater identity and nucleotide sequences having at least 60%, 65%, 70%, 75%, 80%, 85%, 90%, 91%, 92%, 93%, 94%, 95%, 96%, 97%, 98%, 99% or greater identity.

In some embodiments, the polypeptides are encoded by polynucleotides that are optimized for high level expression in the microorganism, such as Shigella, using codons that are preferred in the microorganism. As used herein, a codon that is “optimized for high level expression refers to a codon that is relatively more abundant in the microorganism in comparison with all other codons corresponding to the same amino acid. In some embodiments, at least 10% of the codons are optimized for high level expression. In some embodiments, at least 25%, at least 50%, at least 75%, at least 80%, at least 85%, at least 90%, at least 95%, or at least 99% of the codons are optimized for high level expression.

In some embodiments, the lipid A 1-phosphatase is chromosomally integrated. In some embodiments, the lipid A 1-phosphatase is expressed from a plasmid. In some embodiments, lipid A 1-phosphatase is regulated by an inducible promoter. In some embodiments, the inducible promoter is an osmotically controlled E. coli ompC promoter (P_{ompC}).

In some embodiments, the LpxE is located downstream of an osmotically controlled E. coli ompC promoter (P_{ompC}) and has a nucleotide sequence comprising SEQ ID NO:5.

In some embodiments, the lipid A 1-phosphatase and/or the 3-O deacylase are chromosomally integrated at the attTn7 site in the Gram-negative microorganism, such as Shigella.

In some embodiments, the 3-O deacylase is chromosomally integrated. In some embodiments, the 3-O deacylase is expressed from a plasmid. In some embodiments, the 3-O deacylase is regulated by an inducible promoter. In some embodiments, the inducible promoter is an osmotically controlled E. coli ompC promoter (P_{ompC}).

In some embodiments, the lipid A 1-phosphatase and the 3-O deacylase are expressed from a single nucleic acid cassette. In some embodiments, the lipid A 1-phosphatase and the 3-O deacylase are encoded on separate cassettes.

The genetically modified Gram-negative microorganism, such as Shigella has reduced endotoxicity relative to the parental strain from which it was derived prior to the genetic modification. In some embodiments, administration of the genetically modified Gram-negative microorganism results in production of any of TNF-α, IL-1β, IL-10, IL-6 or other pro-inflammatory cytokines that is dampened compared with administration of its parental strain.

In some embodiments, the Gram-negative microorganism, such as Shigella is attenuated. In some embodiments, the Gram-negative microorganism has more than one attenuating mutations. “Attenuated,” as used herein, refers to the state of the microbe wherein the microbe has been weakened from its wild-type fitness by some form of recombinant or physical manipulation. This includes altering the genotype of the microbe to reduce its ability to cause disease. Methods of attenuation are known in the art. For instance, attenuation may be accomplished by altering (e.g., deleting) native nucleic acid sequences found in the wild type microbe. In some embodiments, the Gram-negative microorganism, such as Shigella has a deletion in one or more genes selected from virG, senA, senB, setAB, iuc and combinations thereof. In some embodiments, the Gram-negative microorganism, such as Shigella has a deletion in virG. In some embodiments, the Gram-negative microorganism, such as Shigella has a deletion in virG, senA, and senB. In some embodiments, the Gram-negative microorganism, such as Shigella has a deletion in virG and iuc. In some embodiments, the Gram-negative microorganism, such as Shigella has a deletion in virG, senA, senB, and setAB.

In some embodiments, the Shigella is selected from S. sonnei and S. flexneri. In some embodiments, the S. sonnei is S. sonnei Moseley. In some embodiments, the S. flexneri is S. flexneri 2a 2457T.

In some embodiments, the S. sonnei strain is strain WRSS1. In some embodiments, the S. sonnei strain is strain WRSs2.

In some embodiments, the S. flexneri 2a strain is strain SC602. In some embodiments, the S. flexneri 2a strain is strain WRSf2G12.

In some embodiments, the lipid A molecules of the Gram-negative microorganism, such as Shigella lack a phosphate at the 1-position. In some embodiments, the lipid A molecules of the Shigella lack an acyl chain at the 3-position.

In some embodiments, the genetically modified microorganism expresses one or more antigens. In some embodiments, the antigen can be native to the genetically modified microorganism. In some embodiments, the antigen is overexpressed in the microorganism in order to improve immunogenicity.

As used herein, “antigen” refers to a biomolecule capable of eliciting an immune response in a host. In some embodiments, an antigen may be a protein, or fragment of a protein. In an exemplary embodiment, the antigen elicits a protective immune response. As used herein, “protective” means that the immune response contributes to the lessening of any symptoms associated with infection of a host with the pathogen the antigen was derived from or designed to elicit a response against. For example, a protective antigen from a pathogen, such as Shigella, may induce an immune response that helps to ameliorate symptoms associated with Shigella infection or reduce the morbidity and mortality associated with infection with the pathogen. The use of the term “protective” in this invention does not necessarily require that the host is completely protected from the effects of the pathogen.

In some embodiments, the antigen to be expressed can be integrated into the chromosome of the genetically modified Gram-negative microorganism, such as Shigella. In some embodiments, the antigen can be expressed on a plasmid.

In some embodiments, the genetically modified microorganism can have a disruption in one or more loci that causes an attenuated phenotype, and the antigen to be expressed can be inserted into said loci.

If antigens or other proteins are overexpressed using plasmids, plasmid stability can be a key factor in the development of high quality attenuated vaccines. Plasmidless bacterial cells tend to accumulate more rapidly than plasmid-bearing cells. One reason for this increased rate of accumulation is that the transcription and translation of plasmid genes imposes a metabolic burden which slows cell growth and gives plasmidless cells a competitive advantage. Furthermore, foreign plasmid gene products are sometimes toxic to the host cell. Thus, it is advantageous for the plasmid to be under some form of selective pressure, in order to ensure that the encoded antigens are properly and efficiently expressed, so that a robust and effective immune response can be achieved.

In some embodiments, the plasmid is selected using a non-antibiotic selection system. For example, the plasmid can encode an essential gene that complements an otherwise lethal deletion/mutation of this locus from the live vector chromosome. It is not necessary that the genetically modified microorganism comprise the complete nucleic acid sequence of the antigen. It is only necessary that the antigen sequence used be capable of eliciting an immune response. The antigen may be one that was not found in that exact form in the parent organism. For example, a sequence coding for an antigen comprising 100 amino acid residues may be transferred in part into a genetically modified microorganism herein so that a peptide comprising only 75, 65, 55, 45, 35, 25, 15, or even 10, amino acid residues is produced by the genetically modified microorganism. Alternatively, if the amino acid sequence of a particular antigen or fragment thereof is known, it may be possible to chemically synthesize the nucleic acid fragment or analog thereof by means of automated nucleic acid sequence synthesizers, PCR, or the like and introduce said nucleic acid sequence into the appropriate copy number vector.

In some embodiments, the antigen is a polypeptide. In some embodiments, the antigen is a fragment or variant sequence. The antigenic fragment can be “free-standing,” or comprised within a larger polypeptide of which they form a part or region, most preferably as a single continuous region.

In some embodiments, the antigenic fragment can include, for example, truncation polypeptides having the amino acid sequence of a polypeptide, except for deletion of a continuous series of residues that includes the amino terminus, or a continuous series of residues that includes the carboxyl terminus or deletion of two continuous series of residues, one including the amino terminus and one including the carboxyl terminus. In some embodiments, fragments are characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, and high antigenic index regions.

The fragment can be of any size. An antigenic fragment is capable of inducing an immune response in a subject or be recognized by a specific antibody. In some embodiments, the fragment corresponds to an amino-terminal truncation mutant. In some embodiments, the number of amino terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to carboxyl-terminal truncation mutant. In some embodiments, the number of carboxyl terminal amino acids missing from the fragment ranges from 1-100 amino acids. In some embodiments, it ranges from 1-75 amino acids, 1-50 amino acids, 1-40 amino acids, 1-30 amino acids, 1-25 amino acids, 1-20 amino acids, 1-15 amino acids, 1-10 amino acids and 1-5 amino acids.

In some embodiments, the fragment corresponds to an internal fragment that lacks both the amino and carboxyl terminal amino acids. In some embodiments, the fragment is 7-200 amino acid residues in length. In some embodiments, the fragment is 10-100 amino acid residues, 15-85 amino acid residues, 25-65 amino acid residues or 30-50 amino acid residues in length. In some embodiments, the fragment is 7 amino acids, 10 amino acids, 12 amino acids, 15 amino acids, 20 amino acids, 25 amino acids, 30 amino acids, 35 amino acids, 40 amino acids, 45 amino acids, 50 amino acids 55 amino acids, 60 amino acids, 80 amino acids or 100 amino acids in length.

In some embodiments, the fragment is at least 50 amino acids, 100 amino acids, 150 amino acids, 200 amino acids or at least 250 amino acids in length. Of course, larger antigenic fragments are also useful according to the present invention, as are fragments corresponding to most, if not all, of the amino acid sequence of a polypeptide from which it is derived.

In some embodiments, the polypeptides have an amino acid sequence at least 80, 85%, 90%, 91%, 92%, 93%, 94%, 95%, 96%, 97%, 98%, 99%, or 100% identical to the polypeptides described herein or antigenic or biologically active fragments thereof. In some embodiments, the variants are those that vary from the reference by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg, or aromatic residues Phe and Tyr. In some embodiments, the polypeptides are variants in which several, 5 to 10, 1 to 5, or 1 to 2 amino acids are substituted, deleted, or added in any combination.

### Therapeutic Methods

In another embodiment, the invention provides a method of inducing an immune response in a subject, comprising administering to the subject an immunologically-effective amount of a genetically modified Gram-negative microorganism, such as a Shigella microorganism or a pharmaceutical composition comprising as provided herein.

In some embodiments, the modified Gram-negative microorganism, such as Shigella microorganism or pharmaceutical composition is orally administered. In some embodiments, the modified Gram-negative microorganism, such as Shigella microorganism or pharmaceutical composition is intranasally administered. In some embodiments, the method comprises administering a combination of genetically modified Gram-negative microorganisms, such as Shigella microorganisms. In some embodiments, the combination comprises a plurality of Shigella strains. In some embodiments, the combination comprises S. sonnei and S. flexneri.

In some embodiments, the subject is first administered the immunologically-effective amount of the modified Gram-negative microorganism, such as Shigella microorganism or pharmaceutical composition as a prime and is subsequently administered an immunologically-effective amount of the modified Gram-negative microorganism, such as Shigella microorganism or pharmaceutical composition as a boost.

In some embodiments, the genetically modified Gram-negative microorganism, such as Shigella microorganism is used as a live vaccine. In other embodiments, the genetically modified Gram-negative microorganism, such as Shigella microorganism may be a species or strain commonly used for a vaccine.

Combinations of genetically modified Gram-negative microorganisms, such as Shigella strains can also be employed, e.g., to make multivalent vaccines.

The term “subject” as used herein, refers to animals, such as mammals. For example, mammals contemplated include humans, primates, dogs, cats, sheep, cattle, goats, pigs, horses, mice, rats, rabbits, guinea pigs, and the like. The terms “subject,” “patient,” and “host” are used interchangeably. In some embodiments, the subject is a human.

As used herein, an “immune response” is the physiological response of the subject's immune system to an immunizing composition. An immune response may include an innate immune response, an adaptive immune response, or both. In one embodiment of the present invention, the immune response is a protective immune response. A protective immune response confers immunological cellular memory upon the subject, with the effect that a secondary exposure to the same or a similar antigen is characterized by one or more of the following characteristics: shorter lag phase than the lag phase resulting from exposure to the selected antigen in the absence of prior exposure to the immunizing composition; production of antibody which continues for a longer period than production of antibody resulting from exposure to the selected antigen in the absence of prior exposure to the immunizing composition; a change in the type and quality of antibody produced in comparison to the type and quality of antibody produced upon exposure to the selected antigen in the absence of prior exposure to the immunizing composition; a shift in class response, with IgG antibodies appearing in higher concentrations and with greater persistence than IgM, than occurs in response to exposure to the selected antigen in the absence of prior exposure to the immunizing composition; an increased average affinity (binding constant) of the antibodies for the antigen in comparison with the average affinity of antibodies for the antigen resulting from exposure to the selected antigen in the absence of prior exposure to the immunizing composition; and/or other characteristics known in the art to characterize a secondary immune response.

In some embodiments, the immune response is sufficient to confer protective immunity upon the subject against a later infection by the pathogen. In some embodiments, the compositions are administered intranasally.

Vaccine strategies are well known in the art and therefore the vaccination strategy encompassed by the invention does not limit the invention in any manner. In certain aspects of the invention, the genetically modified Shigella microorganism vaccine is administered alone in a single application or administered in sequential applications, spaced out over time.

In some embodiments, the subject is further administered an immunologically-effective amount of a pharmaceutical composition comprising an antigen expressed by the genetically modified microorganism.

In some embodiments, a combination of genetically modified microorganisms is administered to the subject. In some embodiments, the combination comprises a plurality of Shigella strains.

In some embodiments, the subject is first administered an effective amount of the genetically modified Gram-negative microorganism, such as Shigella microorganism as a prime and is subsequently administered an immunologically-effective amount of the genetically modified Gram-negative microorganism, such as Shigella microorganism as a boost.

In other aspects of the invention, the genetically modified Gram-negative microorganism, such as Shigella microorganism is administered as a component of a heterologous prime/boost regimen. “Heterologous prime/boost” strategies are 2-phase immunization regimes involving sequential administration (in a priming phase and a boosting phase) of the same antigen in two different vaccine formulations by the same or different route. In particular aspects of the invention drawn to heterologous prime/boost regimens, a mucosal prime/parenteral boost immunization strategy is used. For example, one or more genetically modified Gram-negative microorganisms, such as Shigella microorganism vaccines as taught herein is administered orally or via some other mucosal route such as intranasal and subsequently boosted parentally with a vaccine composition comprising an antigen expressed by the genetically modified microorganism. Subunit or conjugate vaccine compositions are also contemplated as vaccine compositions comprising the antigen which can be used in heterologous prime/boost strategies.

In some embodiments, one or more genetically modified Gram-negative microorganisms, such as Shigella microorganisms or a composition thereof are mucosally administered in a first priming administration, followed, optionally, by a second (or third, fourth, fifth, etc. . . . ) priming administration of the genetically modified Shigella microorganisms or composition thereof from about 2 to about 10 weeks later. In some embodiments, a boosting composition is administered from about 3 to about 12 weeks after the priming administration. In some embodiments, the boosting composition is administered from about 3 to about 6 weeks after the priming administration. In some embodiments, the boosting composition is substantially the same type of composition administered as the priming composition (e.g., a homologous prime/boost regimen).

In some embodiments, the subject is administered the genetically modified Gram-negative microorganism, such as Shigella microorganism as a prime and is subsequently administered an immunologically-effective amount of the genetically modified Gram-negative microorganism, such as Shigella microorganism as a boost. In some embodiments, the subject is boosted one, two, three, four, five, or six times.

In practicing immunization protocols for treatment and/or prevention, an immunologically-effective amount of a genetically modified Gram-negative microorganism, such as Shigella microorganism or composition comprising the microorganism is administered to a subject. As used herein, the term “immunologically-effective amount” means the total amount that is sufficient to show an enhanced immune response in the subject. When “immunologically-effective amount” is applied to an individual therapeutic agent administered alone, the term refers to that therapeutic agent alone. When applied to a combination, the term refers to combined amounts of the ingredients that result in the therapeutic effect, whether administered in combination, serially or simultaneously.

The genetically modified Gram-negative microorganism, such as Shigella microorganism or compositions herein may be administered to warm-blooded mammals of any age. The genetically modified Gram-negative microorganism, such as Shigella microorganism or compositions can be administered as a single dose or multiple priming doses, followed by one or more boosters. For example, a subject can receive a single dose, then be administered a booster dose up to 1 month, 2 months, 3 months, 4 months, 5 months, 6 months, 9 months, 1 year, 2 years, 3 years, 4 years, 5 years, 6 years, 7 years, 8 years, 9 years, or 10 or more years later.

### Pharmaceutical Compositions

In another embodiment, the invention provides a pharmaceutical composition comprising the genetically modified Gram-negative microorganism, such as Shigella microorganism as provided herein and a pharmaceutically acceptable carrier.

A pharmaceutical composition comprising a genetically modified Gram-negative microorganism, such as Shigella microorganism may optionally comprise one or more possible additives, such as carriers, preservatives, stabilizers, adjuvants, and other substances.

In some embodiments, care should be taken when using additives so that the live genetically modified Gram-negative microorganism, such as Shigella microorganism is not killed or have its ability to effectively colonize the host compromised by the use of additives. Stabilizers, such as sucrose, maltose, trehalose, lactose, inositol or monosodium glutamate (MSG), may be added to stabilize the formulation against a variety of conditions, such as temperature variations or a freeze-drying process.

The dosages of a pharmaceutical composition of the invention can and will vary depending on the genetically modified Gram-negative microorganism, such as Shigella microorganism and the intended host, as will be appreciated by one of skill in the art. Generally speaking, the dosage need only be sufficient to elicit a protective immune response in a majority of hosts while also minimizing endotoxicity. Routine experimentation may readily establish the required dosage. In some embodiments, typical initial dosages of vaccine for oral administration could be about 1×10³ to 1×10¹⁰ CFU depending upon the age of the host to be immunized. Administering multiple dosages may also be used as needed to provide the desired level of protective immunity.

The carrier or carriers must be pharmaceutically acceptable in the sense that they are compatible with the therapeutic ingredients and are not unduly deleterious to the recipient thereof. The therapeutic ingredient or ingredients are provided in an amount and frequency necessary to achieve the desired immunological effect.

The mode of administration and dosage forms will affect the therapeutic amounts of the genetically modified microorganism which is desirable and efficacious for the vaccination application. The current application is not limited specifically to oral administration of the vaccine, but can also include parenteral or other mucosal routes including sublingual administration as desired. The genetically modified microorganism Gram-negative microorganism, such as Shigella is delivered in an amount capable of eliciting an immune reaction in which it is effective to increase the patient's immune response to an expressed antigen.

The genetically modified Gram-negative microorganism, such as Shigella microorganism of the present invention may be usefully administered to the host animal with any other suitable pharmacologically or physiologically active agents, e.g., antigenic and/or other biologically active substances.

The genetically modified Gram-negative microorganism, such as Shigella microorganism described herein can be prepared and/or formulated without undue experimentation for administration to a mammal, including humans, as appropriate for the particular application. The pharmaceutical compositions may be manufactured without undue experimentation in a manner that is itself known, e.g., by means of conventional mixing, dissolving, dragee-making, levitating, emulsifying, encapsulating, entrapping, spray-drying, or lyophilizing processes, or any combination thereof.

In one embodiment, the genetically modified Gram-negative microorganism, such as Shigella microorganism is administered mucosally. Suitable routes of administration may include, for example, oral, lingual, sublingual, rectal, transmucosal, nasal, buccal, intrabuccal, intravaginal, or intestinal administration; intravesicular; intraurethral; administration by inhalation; intranasal, or intraocular injections, and optionally in a depot or sustained release formulation. Furthermore, one may administer the compound in a targeted drug delivery system. Combinations of administrative routes are possible.

The dose rate and suitable dosage forms for the genetically modified Gram-negative microorganism, such as Shigella microorganism of the present invention may be readily determined by those of ordinary skill in the art without undue experimentation, by use of conventional antibody titer determination techniques and conventional bioefficacy/biocompatibility protocols. Among other things, the dose rate and suitable dosage forms depend on the particular antigen employed, the desired therapeutic effect, and the desired time span of bioactivity.

In some embodiments, the genetically modified Gram-negative microorganism, such as Shigella microorganism can also be prepared for nasal administration. As used herein, nasal administration includes administering the compound to the mucous membranes of the nasal passage or nasal cavity of the subject. Pharmaceutical compositions for nasal administration of the genetically modified microorganism include therapeutically effective amounts of the genetically modified microorganism prepared by well-known methods to be administered, for example, as a nasal spray, nasal drop, suspension, gel, ointment, cream or powder. Administration of the genetically modified microorganism may also take place using a nasal tampon or nasal sponge.

The compositions may also suitably include one or more preservatives, anti-oxidants, or the like. Some examples of techniques for the formulation and administration of the genetically modified microorganism may be found in Remington: The Science and Practice of Pharmacy, Lippincott Williams & Wilkins Publishing Co., 21^{st} addition, incorporated herein by reference.

In one embodiment, the pharmaceutical compositions contain the genetically modified Gram-negative microorganism, such as Shigella microorganism in an effective amount to achieve their intended purpose. In one embodiment, an effective amount means an amount sufficient to prevent or treat an infection. In one embodiment, to treat means to reduce the development of, inhibit the progression of, or ameliorate the symptoms of a disease in the subject being treated. In one embodiment, to prevent means to administer prophylactically, e.g., in the case wherein in the opinion of the attending physician the subject's background, heredity, environment, occupational history, or the like, give rise to an expectation or increased probability that that subject is at risk of having the disease, even though at the time of diagnosis or administration that subject either does not yet have the disease or is asymptomatic of the disease.

A pharmaceutical composition of the invention may be administered via any suitable route, such as by oral administration or gastric intubation. Additionally, other methods of administering the genetically modified microorganism, such as intravenous, intramuscular, subcutaneous injection, intranasal administration or other parenteral routes, are possible.

In some embodiments, these compositions are formulated for oral administration. Accordingly, these compositions are preferably combined with pharmaceutically acceptable vehicles such as saline, Ringer's solution, dextrose solution, and the like.

Embodiments also encompass kits comprising any one of the compositions above in a suitable aliquot for vaccinating a host in need thereof. In one embodiment, the kit further comprises instructions for use. In other embodiments, the composition is lyophilized such that addition of a hydrating agent (e.g., buffered saline) reconstitutes the composition to generate a vaccine composition ready to administer, preferably orally or intranasally.

### Sample Embodiments

This section describes exemplary embodiments, presented without limitation, as a series of paragraphs, some or all of which may be alphanumerically designated for clarity and efficiency. Each of these paragraphs can be combined with one or more other paragraphs, and/or with disclosure from elsewhere in this application, including the materials incorporated by reference, in any suitable manner. Some of the paragraphs below expressly refer to and further limit other paragraphs, providing without limitation examples of some of the suitable combinations.
1. A genetically modified Gram-negative microorganism with reduced endotoxicity, wherein the Gram-negative microorganism is genetically modified to express a lipid A 1-phosphatase.
2. The genetically modified Gram-negative microorganism of paragraph 1, wherein the Gram-negative microorganism is selected from Shigella, Escherichia coli, Pseudomonas aeruginosa, Salmonella, Vibrio and Yersinia species.
3. The genetically modified Gram-negative microorganism of paragraph 2, wherein the genetically modified Gram-negative microorganism is a Shigella microorganism.
4. The genetically modified microorganism of any of paragraphs 1-3, wherein the lipid A 1-phosphatase is LpxE.
5. The genetically modified microorganism of paragraph 4, wherein the LpxE is from Francisella novicida.
6. The genetically modified microorganism of any of paragraphs 1-5, wherein the lipid A 1-phosphatase is codon optimized for expression in the microorganism.
7. The genetically modified microorganism of any of paragraphs 4-6, wherein the LpxE has an animo acid sequence comprising SEQ ID NO:1.
8. The genetically modified microorganism of any of paragraphs 4-6, wherein the LpxE is encoded by a nucleic acid sequence comprising SEQ ID NO:2.
9. The genetically modified microorganism of any of paragraphs 1-8, wherein the microorganism is genetically modified to express a 3-O deacylase.
10. The genetically modified microorganism of paragraph 9, wherein the 3-O deacylase is PagL.
11. The genetically modified microorganism of paragraph 10, wherein the PagL is from Salmonella enterica.
12. The genetically modified microorganism of paragraph 10, wherein the PagL is codon optimized for expression in the microorganism.
13. The genetically modified microorganism of any of paragraph 11 or 12, wherein the PagL has an animo acid sequence comprising SEQ ID NO:3.
14. The genetically modified microorganism of any of paragraphs 11-14, wherein the PagL is encoded by a nucleic acid sequence comprising SEQ ID NO:4.
15. The genetically modified microorganism of any of paragraphs 1-14, wherein the lipid A 1-phosphatase is chromosomally integrated.
16. The genetically modified microorganism of any of paragraphs 1-14, wherein the lipid A 1-phosphatase is expressed from a plasmid.
17. The genetically modified microorganism of any of paragraphs 1-16, wherein the lipid A 1-phosphatase is regulated by an inducible promoter.
18. The genetically modified microorganism paragraph 17, wherein the inducible promoter is an osmotically controlled E. coli ompC promoter (P_{ompC}).
19. The genetically modified microorganism of any of paragraphs 1-18, wherein the 3-O deacylase is chromosomally integrated.
20. The genetically modified microorganism of any of paragraphs 1-18, wherein the 3-O deacylase is expressed from a plasmid.
21. The genetically modified microorganism of any of paragraphs 1-20, wherein the 3-O deacylase is regulated by an inducible promoter.
22. The genetically modified microorganism of paragraph 21, wherein the inducible promoter is an osmotically controlled E. coli ompC promoter (P_{ompC}).
23. The genetically modified microorganism of any of paragraphs 9-22, wherein the lipid A 1-phosphatase and the 3-O deacylase are expressed from a single cassette.
24. The genetically modified microorganism of any of paragraphs 1-23, wherein the microorganism is attenuated.
25. The genetically modified microorganism of any of paragraphs 1-23, wherein the microorganism has more than one attenuating mutations.
26. The genetically modified microorganism of any of paragraphs 1-25, wherein the microorganism has a deletion in one or more genes selected from virG, senA, senB, setAB, iuc and combinations thereof.
27. The genetically modified microorganism of any of paragraphs 1-26, wherein the microorganism has a deletion in virG.
28. The genetically modified microorganism of any of paragraphs 1-27, wherein the microorganism has a deletion in virG, senA, and senB.
29. The genetically modified microorganism of any of paragraphs 1-27, wherein the microorganism has a deletion in virG and iuc.
30. The genetically modified microorganism of any of paragraphs 1-27, wherein the microorganism has a deletion in virG, senA, senB, and setAB.
31. The genetically modified microorganism of any of paragraphs 1-30, wherein the microorganism is selected from S. sonnei and S. flexneri.
32. The genetically modified microorganism of any of paragraphs 1-31, wherein the S. sonnei is S. sonnei Moseley.
33. The genetically modified microorganism of any of paragraphs 31 or 32, wherein the S. flexneri is S. flexneri 2a 2457T.
34. The genetically modified microorganism of any of paragraphs 1-26, wherein S. sonnei strain WRSS1 is genetically modified.
35. The genetically modified microorganism of any of paragraphs 1-26, wherein S. sonnei strain WRSs2 is genetically modified.
36. The genetically modified microorganism of any of paragraphs 1-26, wherein S. flexneri 2a strain SC602 is genetically modified.
37. The genetically modified microorganism of any of paragraphs 1-24, wherein S. flexneri 2a strain WRSf2G12 is genetically modified.
38. The genetically modified microorganism of any of paragraphs 1-37, wherein the lipid A 1-phosphatase and/or the 3-O deacylase are chromosomally integrated at the attTn7 site in the microorganism.
39. The genetically modified microorganism of any of paragraphs 1-38, wherein lipid A molecules of the microorganism lack a phosphate at the 1-position.
40. The genetically modified microorganism of any of paragraphs 1-39, wherein lipid A molecules of the microorganism lack an acyl chain at the 3-position.
41. A pharmaceutical composition comprising the genetically modified microorganism of any of paragraphs 1-40 and a pharmaceutically acceptable carrier.
42. The pharmaceutical composition of paragraph 41, formulated for oral administration.
43. The pharmaceutical composition of paragraph 41, formulated for intranasal administration.
44. A method of inducing an immune response in a subject, comprising administering to the subject an immunologically-effective amount of the pharmaceutical composition of any of paragraphs 41-43.
45. The method of paragraph 44, wherein the pharmaceutical composition is orally administered.
46. The method of paragraph 44, wherein the pharmaceutical composition is intranasally administered.
47. The method of any of paragraphs 44-46, comprising administering a combination of genetically modified microorganisms.
48. The method of paragraph 47, wherein the combination comprises a plurality of Shigella strains.
49. The method of paragraph 47, wherein the combination comprises S. sonnei and S. flexneri.
50. The method of any of paragraphs 44-49, wherein the subject is first administered the immunologically-effective amount of the pharmaceutical composition as a prime and is subsequently administered an immunologically-effective amount of the pharmaceutical composition as a boost.

Application of the teachings of the present invention to a specific problem is within the capabilities of one having ordinary skill in the art in light of the teaching contained herein. Examples of the compositions and methods of the invention appear in the following non-limiting Examples.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. BECC-Engineered Live-Attenuated Shigella Vaccine Candidates Display Reduced Endotoxicity with Robust Immunogenicity in Mice

In this study, we employed bacterial enzymatic combinatorial chemistry (BECC) to detoxify WRSs2 and WRSf2G12, as well as their respective wild-type strains (Ernst et al., Hajjar, Immunotherapeutic potential of modified lipooligosaccharides/lipid a. United States). Lipid A modifying enzymes LpxE (1-position phosphatase from Francisella) and PagL (3-position deacylase from Salmonella) were ectopically expressed in the Shigella backgrounds. Targeted lipid A dephosphorylation (LpxE), deacylation (PagL), or execution of both modifications (Dual) was confirmed using MALDI-TOF MS. Lipid A dephosphorylation, rather than deacylation, effectively diminished LPS-induced pro-inflammatory immune signaling. Furthermore, deacylation combined with dephosphorylation did not further reduce LPS-mediated signaling. Due to this, only dephosphorylated lipid A mutants (LpxE-modified) were generated in WRSs2 and WRSf2G12, generating WRSs2E and WRSf2G12E. These strains had reduced LPS-mediated pro-inflammatory cytokine production in vitro and severely blunted endotoxemia in vivo, yet they remained as capable as their parental strains to invade epithelia and generate immunogenicity against their O-antigen. Altogether, we characterize two live-attenuated Shigella vaccine candidates (WRSs2E and WRSf2G12E), altered only in their lipid A region, which are greatly detoxified without any consequence to phenotypic traits suggesting that such live-attenuated strains are a promising approach to develop a safe and effective Shigella vaccine.

### Targeted Lipid a Modifications in Shigella

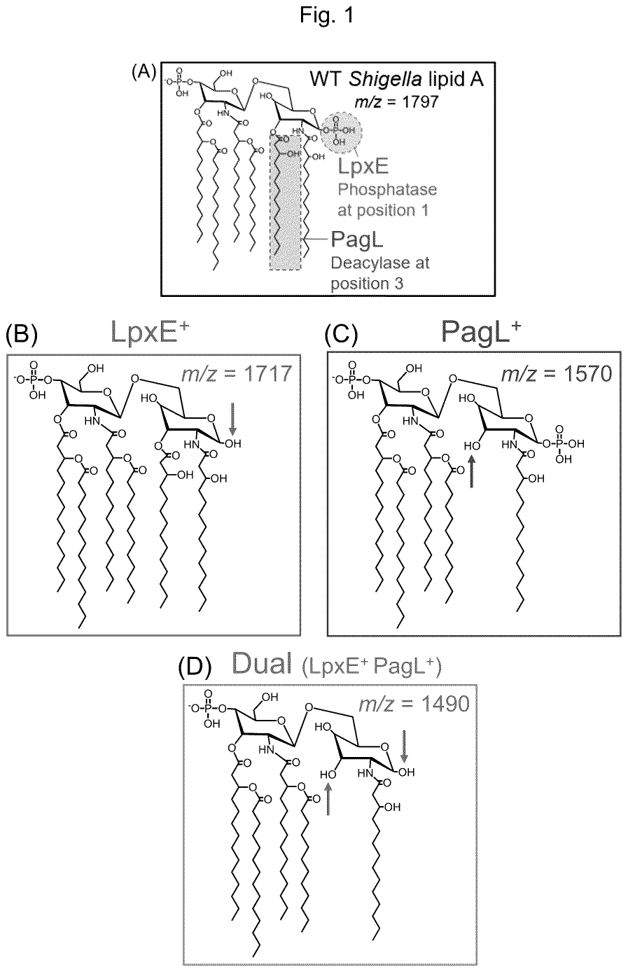

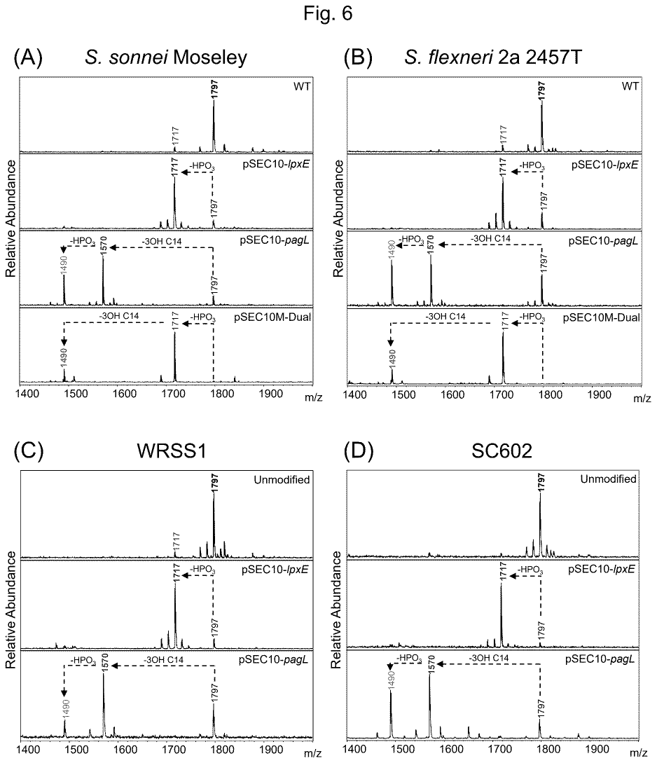

We initially utilized our BECC system to engineer targeted lipid A modifications (FIG. 1) in S. sonnei Moseley and S. flexneri 2a 2457T, the wild-type (WT) strains upon which WRSs2 and WRSf2G12 were derived, respectively. Expression of the lipid A biosynthetic enzymes LpxE and PagL alone, or in combination (termed “Dual”), from the osmotically inducible pSEC10M plasmid resulted in targeted modification of the lipid A structure, which was confirmed via MALDI-TOF MS analysis (FIG. 6A, B). Successful lipid A modification in WT strains prompted the expression of the individual enzymes in first-generation live-attenuated vaccine strains WRSS1 and SC602. Similar to the WT strains, upon plasmid-based expression of BECC constructs, lipid A dephosphorylation and deacylation were observed in WRSS1 and SC602 by MALDI-TOF MS (FIG. 6C, D). In all the Shigella backgrounds tested, PagL-modification was accompanied by spontaneous lipid A dephosphorylation (FIG. 6A-D), suggesting that plasmid-based expression of pagL may induce lipid A phosphatase activity via an unknown mechanism. Despite this, the MALDI-TOF MS data suggested that targeted lipid A modifications could be achieved in both WT and genetically attenuated strains of Shigella.

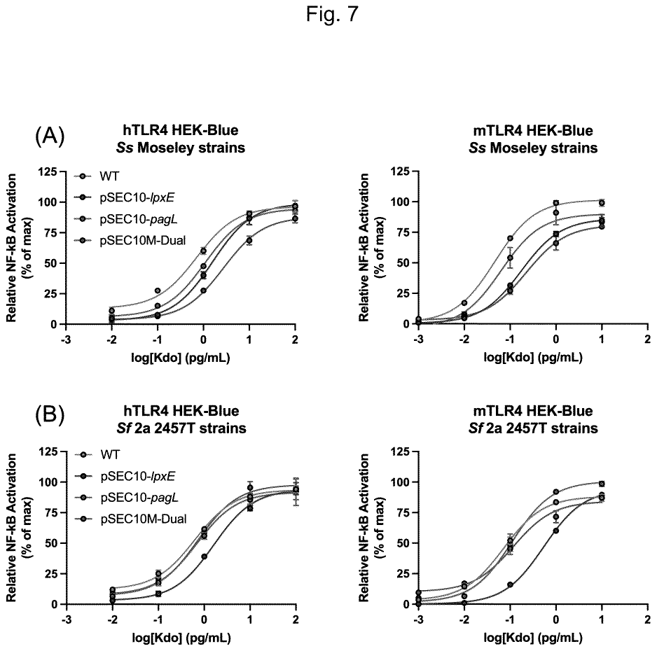

We next assessed whether these targeted lipid A modifications affected the immunostimulatory capacity of Shigella LPS. Purified LPS from Moseley and 2457T was normalized to keto-deoxy-octanoate (Kdo), a conserved sugar within the core oligosaccharide, and used to stimulate NF-κB reporter cells. For LPS from Moseley, stimulation with LpxE- and Dual-modified LPS resulted in a pronounced reduction in NF-κB signaling compared to WT LPS, whereas stimulation with PagL-modified LPS generated comparable NF-κB signaling to that of WT LPS (FIG. 7A). For LPS from 2457T, however, only the LpxE-modification reduced NF-κB signaling compared to WT LPS (FIG. 7B). This suggested that lipid A dephosphorylation, rather than deacylation, was the most promising modification for reduced endotoxicity, and therefore, only the LpxE- and Dual-modification were pursued for the remainder of the study.

To remove the need for ectopic plasmid expression of the lipid A modification enzymes, we utilized Tn7 transposition to insert the lpxE and Dual gene cassettes into the chromosome of Moseley and 2457T. In this approach, insertion occurs exclusively at the attTn7 site located at the 3′ end of the highly conserved glmS gene and is known not to disrupt any chromosomal genes or the bacterial fitness (McKenzie et al., BMC Microbiology, (2006), 6:39). Using the same approach, second-generation vaccine strains WRSs2 and WRSf2G12 were also chromosomally integrated with lpxE; however, the Dual gene cassette could not be chromosomally integrated into these specific vaccine strains. For the remainder of the study, these chromosomally integrated strains were utilized. Moseley and 2457T are designated with LpxE⁺ or Dual to indicate what chromosomal integration they contain. Vaccine strains WRSs2 and WRSf2G12 chromosomally integrated with lpxE are designated as WRSs2E and WRSf2G12E, respectively.

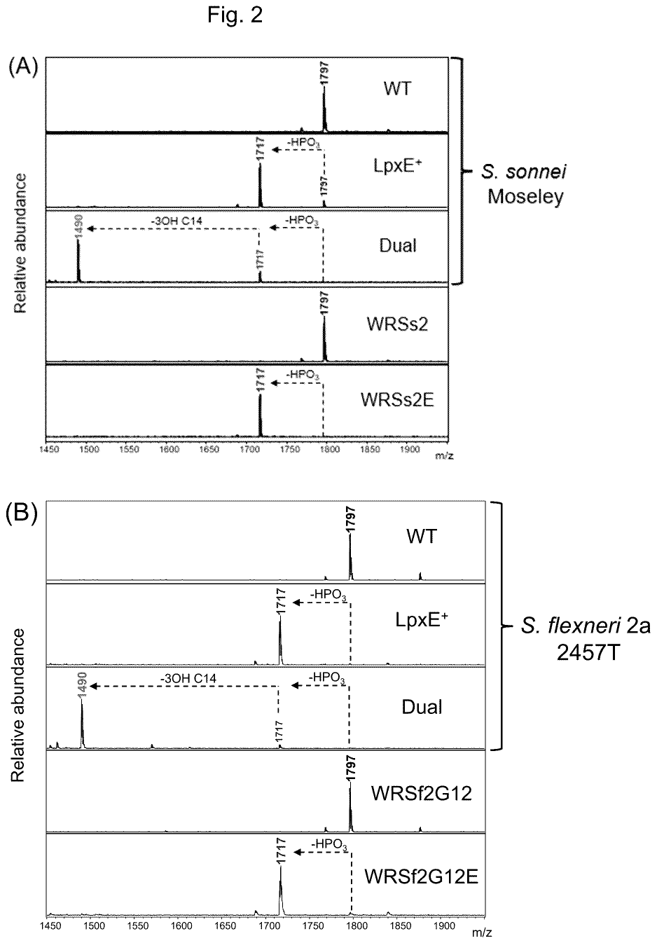

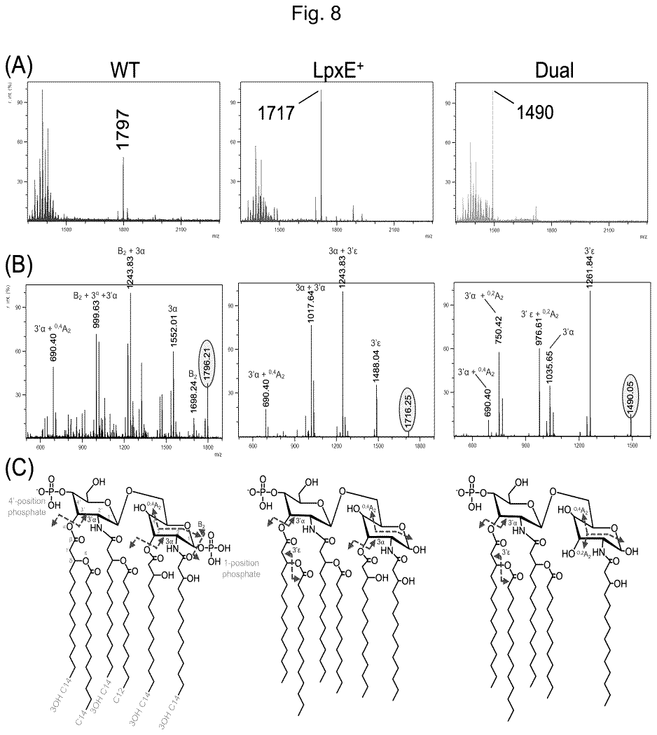

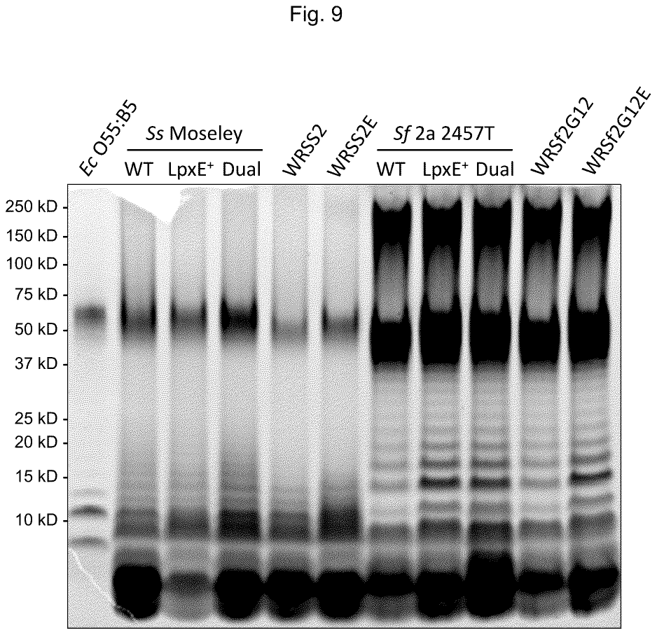

Using MALDI-TOF MS, we confirmed that chromosomal expression of lpxE and Dual resulted in lipid A modification across the various Shigella backgrounds (FIG. 2). Site-specificity of the lipid A modifications was confirmed via MALDI-TOF MS/MS using the FLATⁿ approach (Yang et al., Anal Chem, (2022), 94:7460-7465). WT, LpxE-, and Dual-modified LPS each synthesized their expected base peaks at m/z 1797, 1717, and 1490, respectively (FIG. 8A), which were selected as the precursor ions for fragmentation (FIG. 8B, circled in red). The large cluster of spectral peaks at m/z less than 1490 represents cardiolipin (FIG. 8A), another phospholipid that resides in the outer leaflet along with lipid A. Spectral peaks representing fragments from the precursor ions (FIG. 8B) were mapped to the proposed chemical structures of WT, LpxE-, and Dual-modified Shigella lipid A (FIG. 8C). Determination of the location of the terminal phosphate modification at the 1-position was confirmed by the fragment ion still present at m/z 690 in the LpxE-modified structure, and the deacylation modification at the 3-position was confirmed by the ions generated from the ^{0.2}A₂ cross-ring cleavage event (FIG. 8C). Furthermore, SDS-PAGE and subsequent Pro-Q Emerald 300 staining of LPS extracted from the LpxE- or Dual-modified strains showed no alterations to the O-antigen region compared to their parental counterparts, suggesting the modifications were specific to the lipid A region (FIG. 9).

BECC-Modified Shigella Strains Phenocopy their Isogenic Parental Strains

Live-attenuated Shigella strains have fundamental requirements that enable them to function as effective oral vaccines. First, large-scale production requires efficient growth in culture. Secondly, these strains must be capable of invading gut epithelia, a step during infection that elicits the host immune response required for mucosal immunity (Barry et al., Nature Reviews Gastroenterology & Hepatology, (2013), 10: 245-255). Lastly, host defense responses must be unaltered, such as the secretion of CXCL8 from the gut epithelia, which recruits polymorphonuclear leukocytes to the site of infection to clear the bacteria (Schroeder et al., Clin Microbiol Rev, (2008), 21:134-56). Thus, any limitation with respect to growth, invasion, or the host response would render our BECC-modified Shigella strains unsuitable to be used as vaccine candidates.

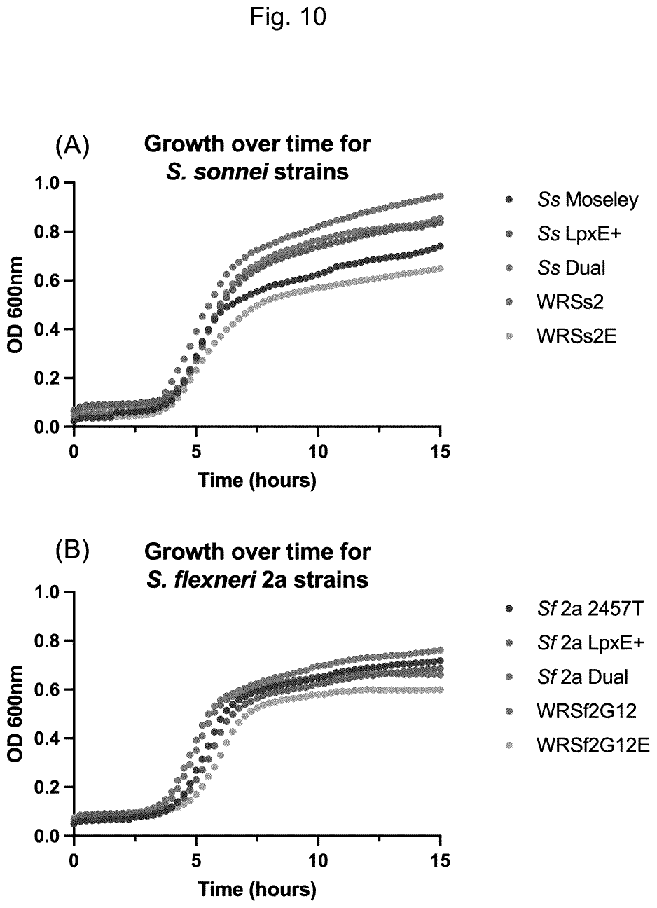

To evaluate the capacity to grow in culture, we assayed the growth kinetics of our BECC-modified Shigella and compared it to their isogenic parental strains. Over the course of 15 hours, BECC-modified Shigella showed minimal growth alterations as compared to their unmodified counterparts (FIG. 10), suggesting BECC-modification had minimal impact on the capacity of the Shigella strains to grow in vitro.

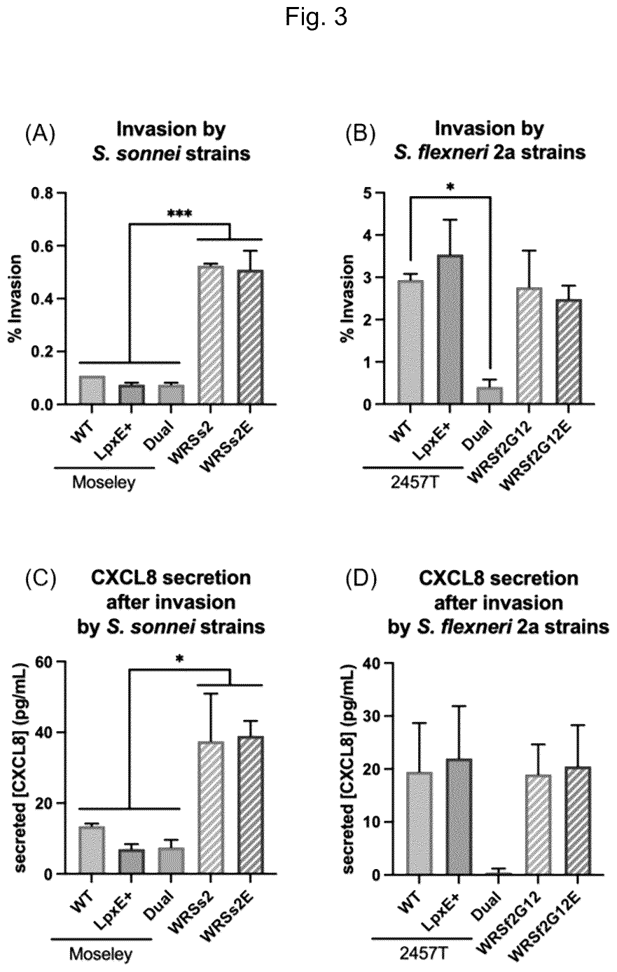

To evaluate the invasion of epithelia by Shigella (intracellular bacterial burden recovered as a percentage of the inoculum), we employed a gentamicin protection assay (Barnoy et al., Vaccine, (2010), 28:1642-1654; Bedford et al., Gut Microbes, (2011), 2:244-251; Ranallo et al., Vaccine, (2012), 30:5159-5171). The BECC-modified S. sonnei strains invaded similarly to their parental strains. As found previously, WRSs2 strains invaded the epithelial cells significantly more than the Moseley strains (FIG. 3A) (Barnoy et al., Vaccine, (2010), 28:1642-1654). For S. flexneri 2a strains, while LpxE-modification did not impact invasiveness, Dual-modification resulted in a significantly lower invasion than the WT (FIG. 3B), likely due to a high frequency of invasion plasmid loss compared to the other S. flexneri 2a strains (data not shown). Beyond invasion, we also measured CXCL8 concentrations in the supernatant to assess the host response to infection, which showed the same pattern as invasion (FIG. 3C, D). This suggested that, except for the Dual-modification in 257T, the targeted lipid A modifications did not impact the phenotypic traits of Shigella, such as growth, invasion of gut epithelia, or the host response to infection.

### Lipid a Modifications Reduced Endotoxicity In Vitro and In Vivo

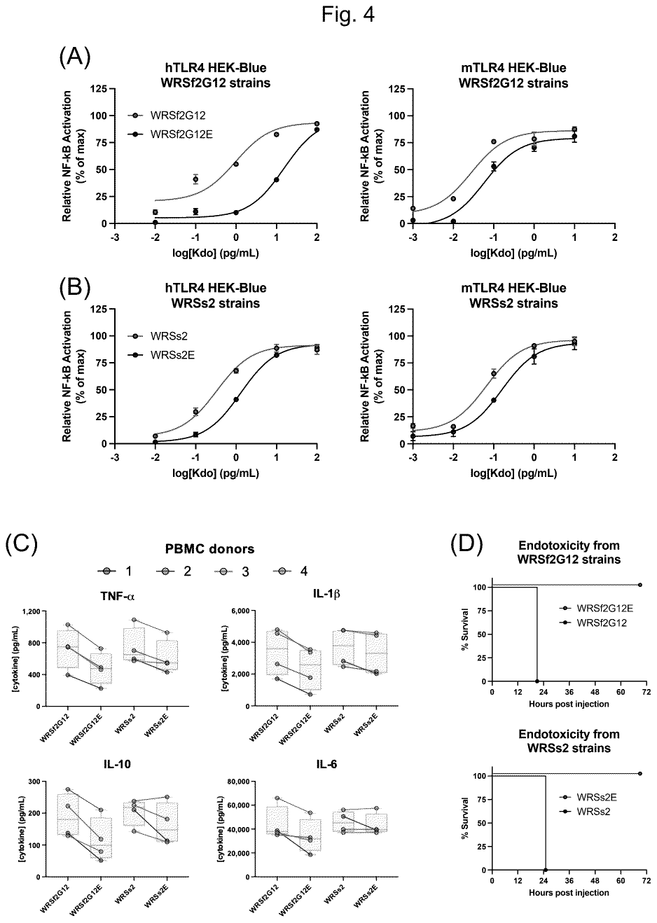

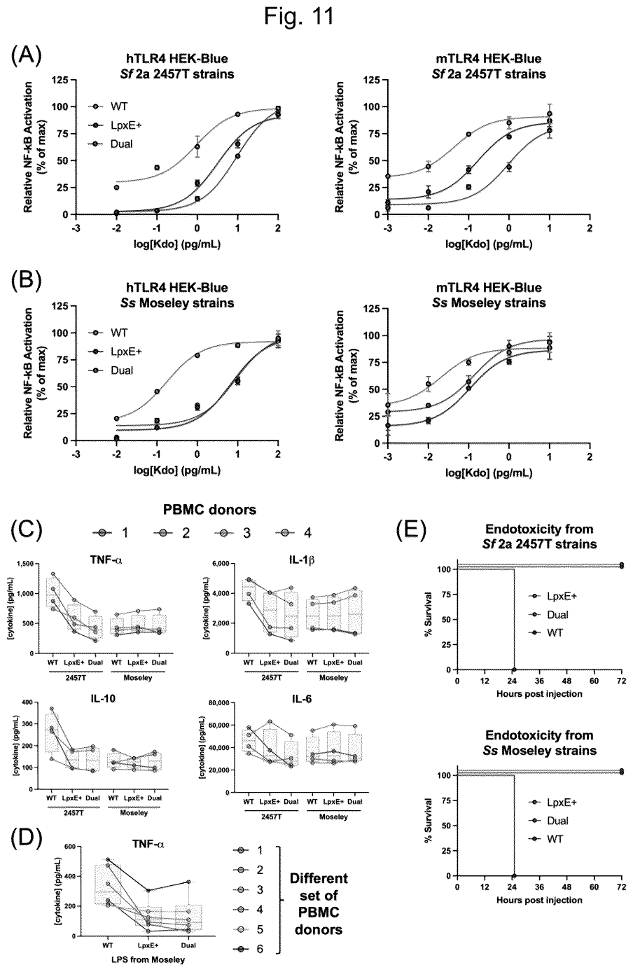

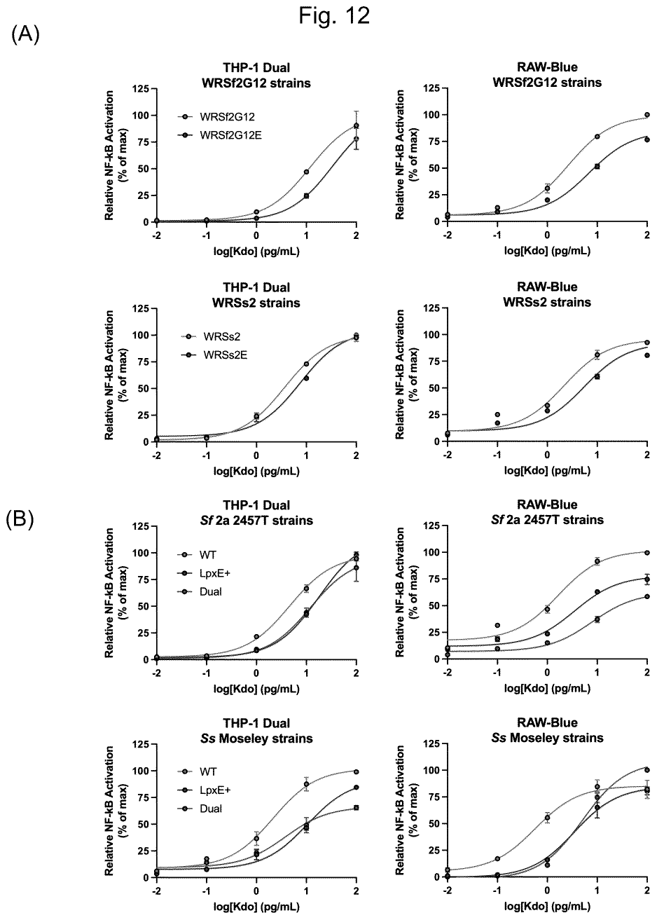

Since detoxification of Shigella lipid A was the primary objective of this study, we determined the level of endotoxicity using purified LPS from the chromosomally integrated strains. HEK-Blue cells containing an NF-κB reporter and stably expressing either the human or mouse orthologs of TLR4/MD-2/CD-14 were stimulated with Kdo normalized LPS. Stimulation with LPS from WRSs2E or WRSf2G12E displayed reduced NF-κB signaling compared to LPS from their respective parental strain (FIG. 4A, B). Similar results were observed upon stimulating HEK-Blue cells with LPS from BECC-modified 2457T and Moseley (FIG. 10A, B) or upon stimulation of macrophage NF-κB reporter cells that express endogenous levels of TLR4/MD-2/CD-14 (FIG. 11). In both cases, LpxE- and Dual-modified LPS blunted NF-κB signaling to a similar degree (FIGS. 11A, 11B, and 12), suggesting lipid A dephosphorylation alone was sufficient to reduce the LPS-mediated TLR4 signaling.

To examine the LPS-induced TLR4 signaling in primary cells, we stimulated human peripheral blood monocytes (PBMCs) with LPS and assayed the supernatant for cytokine production. Notably, production of TNF-α, IL-1β, IL-10, and IL-6 was dampened upon stimulation with LPS from WRSs2E or WRSf2G12E compared to LPS from their parental strains, albeit the reduction in WRSs2E was more modest than that observed for WRSf2G12E (FIG. 4C). Similarly, stimulation of PBMCs with LPS from 2457T had lower cytokine production for BECC-modified LPS compared to WT LPS (FIG. 11C). BECC-modified LPS from Moseley demonstrated similar cytokine levels to that of BECC-modified LPS from 2457T; however, WT LPS from Moseley was less stimulatory than expected (FIG. 11C). A separate experiment showed that LpxE- and Dual-modified LPS from Moseley display reduced induction of TNF-α from PBMCs, and to a similar degree, compared to WT LPS (FIG. 11D). This confirmed that dephosphorylated Shigella lipid A does indeed reduce pro-inflammatory responses across all the Shigella backgrounds.

To confirm that the BECC-modified LPS was also detoxified in vivo, we employed an acute murine endotoxicity study whereby a normally lethal dose of LPS was injected intraperitoneally into mice, and their health status monitored over the course of 72 hours. Whereas injection of LPS from WRSf2G12 or WRSs2 was lethal by 24 hours post-injection, all mice receiving the same dose of LPS from WRSf2G12E or WRSs2E survived (FIG. 4D). Similarly, mice receiving WT LPS from 2457T or Moseley succumbed to acute endotoxemia whereas mice receiving BECC-modified LPS from 2457T or Moseley survived (FIG. 11E). This, combined with the NF-κB reporter and PBMCs data, suggested that dephosphorylation of lipid A was sufficient to reduce endotoxicity both in vitro and in vivo.

### LpxE-Modification Did not Compromise the Immunogenicity of the Shigella Vaccine Strains

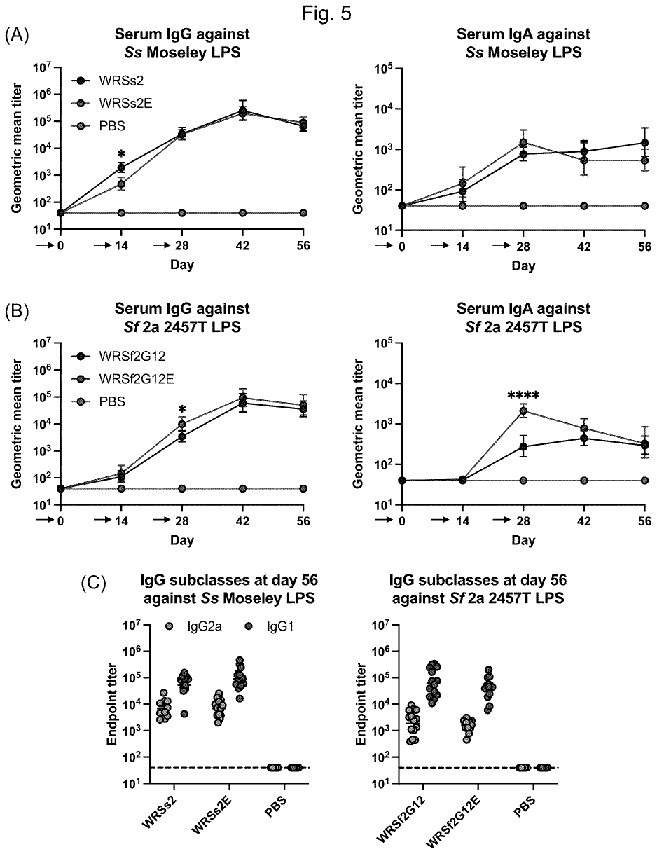

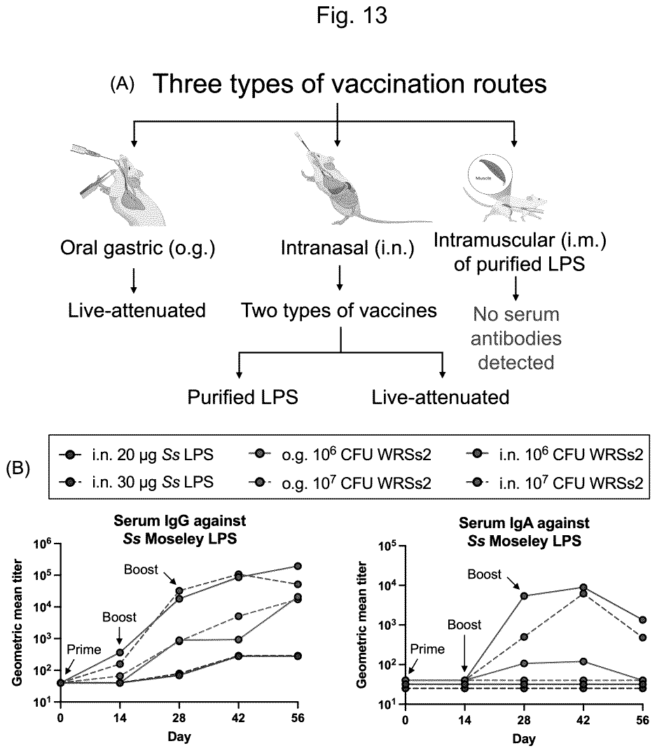

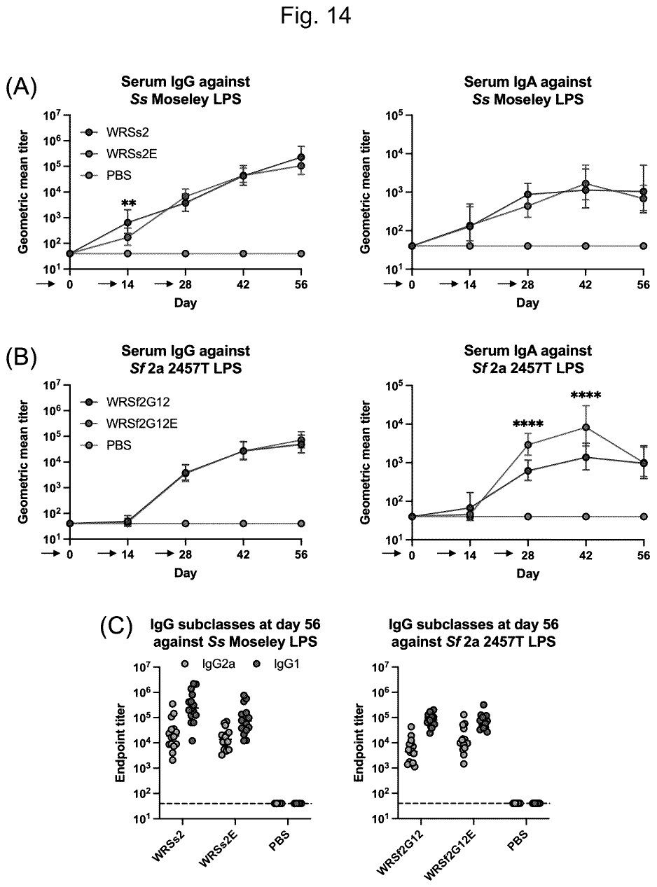

To evaluate their potential use as vaccine candidates, we compared the immunological response of WRSs2E and WRSf2G12E to their parental strains in a mouse model. As mice do not experience diarrheal episodes from ingestion of Shigella, we evaluated vaccine efficacy through the generation of Shigella-specific immunological responses. Using three routes of vaccination (oral, intranasal, and intramuscular) and two different types of vaccines (live bacteria or purified LPS) (FIG. 13A), we determined that intranasal administration of live bacteria at Day 0, 14, and 28 (prime-boost-boost, respectively) generated the most reliable serum antibody response against serotype-specific LPS (FIG. 13B). Using this intranasal approach and the same vaccination scheme of prime-boost-boost at Day 0, 14, and 28, respectively, we showed that vaccination with WRSs2E or WRSf2G12E elicited strong serum IgG and IgA responses against serotype-specific LPS, that mimicked the response from their parental strains (FIG. 5A, B). Although statistically significant differences in antibody titers were observed between the parental and LpxE-modified strains at specific time points, these differences did not remain throughout the entire vaccine study. At Day 56, four weeks after the final vaccine dose was delivered, the skewing of IgG subclasses 2a and 1 was similar for WRSs2E and WRSf2G12E compared to their parental strains (FIG. 5C). The same patterns were observed in a second independent vaccine study (FIG. 14). Ultimately, this data supports the notion that WRSs2E and WRSf2G12E promote a similar adaptive immune response as their parental strains.

### Discussion

Despite significant advances in our understanding of Shigella pathogenesis and the development of many vaccine candidates (MacLennan et al., Vaccines (Basel), (2022), 10(9)), to date, there is no FDA-licensed vaccine available. This study utilized second-generation vaccine candidates WRSs2 and WRSf2G12, whose first-generation strains were generally well tolerated in clinical trials but deemed too reactogenic to be considered safe for general use (Katz et al., Infection and Immunity, (2004), 72:923-930; Frenck et al., Vaccine, (2018), 36:4880-488). These second-generation vaccine strains contain a suite of genetic manipulations that remove known enterotoxins and reduce the spread of the bacterium across gut epithelia (Hartman et al., Infect Immun, (1998), 66:4572-6; Barnoy et al., Vaccine, (2010), 28:1642-1654; Ranallo et al., Vaccine, (2012), 30:5159-5171). In this study, we describe the engineering and characterization of an additional modification, specifically the dephosphorylation of their lipid A structure, to generate WRSs2E and WRSf2G12E, which have reduced endotoxicity while retaining the same phenotypic traits as their isogenic parental strains.

The lipid A modifications engineered in this study utilized the BECC approach whereby prior identification of lipid A modifying enzymes from a variety of Gram-negative bacteria then enabled the expression of select enzymes within a bacterium of interest. Previously, the BECC system had been employed to generate custom-designed lipid A molecules in Yersinia (Gregg et al., mBio, (2017), 8:e00492-17; Gregg et al., Vaccine, (2018), 36:4023-4031; Haupt et al., Vaccine, (2021), 39:5205-5213; Zacharia et al., Vaccine, (2021), 39:292-302; Alexander-Floyd et al., Infect Immun, (2022), 90:e0020122; Harberts et al., Infect Immun, (2022), 90: e0020822; Haupt et al., Sci Rep, (2023), 13:715); however, this study extends its use to Shigella species, suggesting it is applicable to a variety of Gram-negative bacteria. More specifically, we showed that BECC enabled robust lipid A modification in both WT and vaccine strains of Shigella. Whereas multiple lipid A related spectral peaks were present upon plasmid-based expression of BECC constructs (FIG. 6), chromosomal expression generated a single spectral peak (FIG. 2) indicative of more complete lipid A modification on the outer membrane. This demonstrates a newfound approach for the expression of BECC enzymes since here we showed that chromosomally expressing strains are both stable and robustly modify their lipid A, all without the requirement for antibiotic selection.

Furthermore, to function as an effective oral vaccine, specific phenotypic requirements are required that may be altered upon engineering modified lipid A strains. We showed that dephosphorylation of lipid A had no impact on invasion or growth; however, Dual-modification (both dephosphorylation and deacylation) caused S. flexneri 2a 2457T to lose its invasion plasmid more frequently, likely a consequence of increased membrane stress. This is further supported by the inability to chromosomally integrate the Dual construct into WRSs2 or WRSf2G12, suggesting a limit to the degree to which lipid A can be modified in already genetically attenuated strains.

Additionally, we showed that dephosphorylation of lipid A at the 1-position via LpxE was sufficient to effectively blunt the LPS-induced pro-inflammatory response from both human and murine immune cells. This is analogous to other contexts, such as sepsis, where human alkaline phosphatase dephosphorylates LPS to reduce inflammatory signaling (Pettengill et al., PLoS One, (2017), 12: e0175936). Traditionally, however, it is thought that deacylation of lipid A reduces LPS-induced TLR4/MD-2 signaling as tetra- and penta-acylated structures are generally less immunostimulatory than hexa-acylated structures (Scott et al., Biochimica et Biophysica Acta (BBA)—Molecular and Cell Biology of Lipids, (2017), 1862:1439-1450). Here, we showed that PagL-mediated lipid A deacylation in Shigella at the 3-position did not greatly reduce LPS-mediated signaling (FIG. 7). Conversely, it has been shown that different penta-acylated Shigella lipid A structures, other than the one generated in this study, do in fact diminish LPS-induced signaling. Rossi et. al., showed that an htrB mutant in S. sonnei, whose lipid A lacks the secondary acyl chain at the 2′-position, reduced TLR4 signaling in NF-κB reporter cells; however, the same mutation in S. flexneri 2a led to a compensatory C16:1 addition and no reduction in signaling compared to WT LPS (Rossi et al., Journal of Biological Chemistry, (2014), 289:24922-24935). This suggested that detoxification of Shigella lipid A via deacylation is site-specific. Furthermore, it emphasizes the complexity of achieving complete lipid A deacylation via genetic manipulation in Shigella. For instance, Shigella contains two msbB genes (both encoding MsbB/LpxM), one chromosomal and one on its invasion plasmid (D'Hauteville et al., The Journal of Immunology, (2002), 168:5240-5251; Ranallo et al., Infect Immun, (2010), 78:400-12). Additionally, Shigella can induce lpxP (encodes LpxP, C16:1 acylase) in the absence of htrB (encodes HtrB/LpxL) under stress-inducing conditions (Simpson et al., Nature Reviews Microbiology, (2019), 17:403-416; Carty et al., J Biol Chem, (1999), 274:9677-85; Needham et al., Nat Rev Microbiol, (2013), 11:467-81). Altogether, this genetic redundancy of lipid A biosynthetic enzymes in Shigella highlights its drive to maintain hexa-acylated lipid A. Using BECC avoids this complication, as it introduces exogenous lipid A modifying enzymes and prevents induction of compensatory mechanisms that revert its lipid A back to the hexa-acylated state. This is emphasized in the present study as primarily a single spectral peak was observed in the MALDI-TOF MS spectra upon expression of BECC constructs across the various Shigella backgrounds (FIG. 2), suggesting that the outer membrane contains predominantly the targeted lipid A structure without any compensatory lipid A modifications.

Despite remodeling of the lipid A region of LPS in the WRSs2E and WRSf2G12E, murine vaccination with these strains generated similar immune responses to their isogenic parental strains (FIGS. 5 and 14). This suggested that serotype-specific immunity in response to vaccination with these strains was unaffected by BECC modification. Since Shigella does not cause diarrheal disease upon ingestion in rodents, only immunological responses were evaluated in this study; however, the first-generation variants of WRSs2 (WRSS1) and WRSf2G12 (SC602) have shown efficacy against shigellosis in humans (Coster et al., Infection and Immunity, (1999), 67:3437-3443; Pitisuttithum et al., Clinical and Vaccine Immunology, (2016), 23:564-575). Since LpxE-modification abrogates the toxic effects of LPS and does not appear to impact immunogenicity, this supports the notion that oral vaccination in humans with WRSs2E and WRSf2G12E would have reduced reactogenicity while maintaining robust immunogenicity and protection against shigellosis.

Overall, the need for a Shigella vaccine remains a priority. While it has been proposed that endemic Shigella can be controlled via public health efforts, the low infectious dose combined with its capacity to acquire extreme drug resistance has bolstered vaccination as a promising option to control the spread of this pathogen. To date, live-attenuated Shigella vaccines have shown immunological success in humans and, in some cases, protection against virulent challenges; this suggests that optimization of current live-attenuated vaccine candidates is a promising approach. A significant drawback with live-attenuated vaccine candidates, however, is the adverse effects from ingestion of high doses of bacteria containing the immunostimulatory hexa-acylated Shigella LPS. The BECC-modified vaccine strains of Shigella developed in this study, namely WRSs2E and WRSf2G12E, contain detoxified LPS and thus have promise to be better tolerated, safer, live-attenuated vaccine candidates.

### Methods

### Bacterial Strains and Growth Conditions

Bacterial strains used in these studies are listed in Table 1. Bacteria were grown at 30° C. or 37° C. in Lysogenic Broth (Teknova) and Tryptic Soy broth (TSB) or on Tryptic Soy agar (TSA) (Becton Dickinson) supplemented with 50 μg/mL neomycin (Sigma) or 60 μg/mL carbenicillin (Sigma) as needed. All strains were supplemented with 1 mM MgCl₂ to repress the PhoPQ two-component regulatory system. Growth curves were performed in a flat-bottom 96-well uncoated sterile plate (Costar) and recorded using a Cerillo Stratus instrument (Cerillo). Each well was inoculated with 10⁵ CFU in 200 mL TSB and incubated with shaking (180 RPM), at 37° C. for 15 hours. Absorbance readings at 600 nm were taken every 15 minutes.

TABLE 1
Bacterial strains used in this study

| | | | Modified | |
|---|---|---|---|---|
| Strain | Description | Genotype | lipid A | Ref. |
| S. sonnei | S. sonnei parent strain | Wild-type | − | ¹ |
| Moseley | | | | |
| S. flexneri 2a | S. flexneri 2a parent strain | Wild-type | − | ² |
| 2457T | | | | |
| WRSS1 | 1^{st} generation attenuated vaccine | ΔvirG | − | 3 |
| | strain of S. sonnei | | | |
| WRSs2 | 2^{nd} generation attenuated | ΔvirG, senA, senB | − | 1, 4-8 |
| | vaccine strain of S. sonnei | | | |
| SC602 | 1^{st} generation attenuated vaccine | ΔvirG, iuc | − | 9, 10 |
| | strain of S. flexneri 2a | | | |
| WRSf2G12 | 2^{nd} generation attenuated | ΔvirG, senA, senB, setAB | − | 6, 11 |
| | vaccine strain of S. flexneri 2a | | | |
| E. coli S17-1 | Mobilizes oriT-carrying | recA, pro, RP4-2 Tet::Mu- | − | ¹² |
| | plasmids for conjugal | Kan::Tn7 | | |
| | transfer | | | |
| E. coli S17-1 | For conjugation of pGRG36- | pGRG36::P_{ompC}-lpxE | − | This |
| pGRG36-lpxE | lpxE | | | study |
| E. coli S17-1 | For conjugation of pGRG36- | pGRG36::P_{ompC}-Dual | − | This |
| pGRG36-Dual | Dual | | | study |
| E. coli DH5α | For plasmid propagation | F⁻ Φ80lacZΔM15 Δ(lacZYA- | − | Thermo |
| | | argF)U169 recA1 endA1 hsdR17 | | Fisher |
| | | (rₖ⁻, mₖ⁺) phoA supE44 thi- | | |
| | | 1 gyrA96 relA1 λ⁻ | | |
| E. coli TOP10 | For plasmid propagation | F− mcrA Δ(mrr-hsdRMS- | − | Thermo |
| | | mcrBC) Φ80lacZΔM15 | | Fisher |
| | | ΔlacX74 recA1 araD139 | | |
| | | Δ(araleu)7697 galU galK rpsL | | |
| | | (StrR) endA1 nupG | | |
| S. sonnei | Wild-type strain, plasmid- | pSEC10M::P_{ompC}-pagL | + | This |
| pSEC10M-pagL | based expression of pagL | | | study |
| | under control of P_{ompC} | | | |
| S. sonnei | Wild-type strain, plasmid- | pSEC10M::P_{ompC}-lpxE | + | This |
| pSEC10M-lpxE | based expression of lpxE | | | study |
| | under control of P_{ompC} | | | |
| S. sonnei | Wild-type strain, plasmid- | pSEC10M::P_{ompC}-lpxE-pagL | + | This |
| pSEC10M-Dual | based expression of Dual | | | study |
| | under control of P_{ompC} | | | |
| S. flexneri 2a | Wild-type strain, plasmid- | pSEC10M::P_{ompC}-pagL | + | This |
| pSEC10M-pagL | based expression of pagL | | | study |
| | under control of P_{ompC} | | | |
| S. flexneri 2a | Wild-type strain, plasmid- | pSEC10M::P_{ompC}-lpxE | + | This |
| pSEC10M-lpxE | based expression of lpxE | | | study |
| | under control of P_{ompC} | | | |
| S. flexneri 2a | Wild-type strain, plasmid- | pSEC10M::P_{ompC}-lpxE-pagL | + | This |
| pSEC10M-Dual | based expression of Dual | | | study |
| | under control of P_{ompC} | | | |
| WRSs1 | Attenuated 1^{st} generation | ΔvirG; pSEC10M::P_{ompC}-pagL | + | This |
| pSEC10M-pagL | vaccine strain of S. sonnei, | | | study |
| | plasmid-based expression of | | | |
| | pagL under control of P_{ompC} | | | |
| WRSs1 | Attenuated 1^{st} generation | ΔvirG; pSEC10M::P_{ompC}-lpxE | + | This |
| pSEC10M-lpxE | vaccine strain of S. sonnei, | | | study |
| | plasmid-based expression of | | | |
| | lpxE under control of P_{ompC} | | | |
| SC602 | Attenuated 1^{st} generation | ΔvirG, iuc; pSEC10M::P_{ompC}- | + | This |
| pSEC10M-pagL | vaccine strain of S. flexneri | pagL | | study |
| | 2a, plasmid-based expression | | | |
| | of pagL under control of | | | |
| | P_{ompC} | | | |
| SC602 | Attenuated 1^{st} generation | ΔvirG, iuc; pSEC10M::P_{ompC}- | + | This |
| pSEC10M-lpxE | vaccine strain of S. flexneri | lpxE | | study |
| | 2a, plasmid-based expression | | | |
| | of lpxE under control of | | | |
| | P_{ompC} | | | |
| S. sonnei LpxE⁺ | Wild-type strain, | attTn7::P_{ompC}-lpxE | + | This |
| | chromosomal expression of | | | study |
| | lpxE under control of P_{ompC} | | | |
| | from attTn7 site from attTn7 | | | |
| | site | | | |
| S. sonnei Dual | Wild-type strain, | attTn7::P_{ompC}-lpxE-pagL | + | This |
| | chromosomal expression of | | | study |
| | Dual under control of P_{ompC} | | | |
| | from attTn7 site | | | |
| S. flexneri 2a | Wild-type strain, | attTn7::P_{ompC}-lpxE | + | This |
| LpxE⁺ | chromosomal expression of | | | study |
| | lpxE under control of P_{ompC} | | | |
| | from attTn7 site | | | |
| S. flexneri 2a | Wild-type strain, | attTn7::P_{ompC}-lpxE-pagL | | |
| Dual | chromosomal expression of | | + | This |
| | Dual under control of P_{ompC} | | | study |
| | from attTn7 site | | | |
| WRSs2E | Attenuated 2^{nd} generation | ΔvirG, senA, senB; | + | This |
| | vaccine strain of S. sonnei, | attTn7::P_{ompC}-lpxE | | study |
| | chromosomal based | | | |
| | expression of lpxE under | | | |
| | control of P_{ompC} from attTn7 | | | |
| | site | | | |
| WRSf2G12E | Attenuated 2^{nd} generation | ΔvirG, senA, senB, setAB; | + | This |
| | vaccine strain of S. flexneri | attTn7::P_{ompC}-lpxE | | study |
| | 2a, chromosomal based | | | |
| | expression of lpxE under | | | |
| | control of P_{ompC} from attTn7 | | | |
| | site | | | |

Molecular Genetic Techniques Standard DNA techniques, liquid media, and agar plates were used as described (Sambrook et al., (2001), Cold Spring Harbor, N.Y.,: Cold Spring Harbor Laboratory Press). Restriction endonucleases and T4 DNA ligase were used as recommended by the manufacturer (New England Biolabs). DNA used for cloning purposes was PCR amplified using 10 mM dNTP mix (Thermo Scientific) and high-fidelity DNA polymerases Q5 (New England Biolabs) or Pfu ultra 11 fusion HS (Agilent) according to manufacturer's instructions. Go-Taq polymerase (Promega) was used for genetic screening. DNA oligonucleotides were obtained from Integrated DNA Technologies and are listed in Table 2. All plasmid constructs (Table S3) were confirmed by double-stranded sequencing (Azenta) and maintained in E. coli DH5a or E. coli TOP10 (ThermoFisher).

TABLE 2
Primers used in this study

| Primer | Sequence 5′ → 3′ | Reference |
|---|---|---|
| ospD3-F | TACACGTCCATTATGCAAGGCT | 13 |
| | (SEQ ID NO: 6) | |
| ospD3-R | TGCCATCAGTAAATTTAATCCC | |
| | ATC (SEQ ID NO: 7) | |
| ipaB-F | CACAGCATCTGCTGAACAGC | This study |
| | (SEQ ID NO: 8) | |
| ipaB-R | CAGCAGAAGCGACACTTCCT | |
| | (SEQ ID NO: 9) | |
| lpxE-F | GGATCCATGCTCAAACAGACC | This study |
| | (SEQ ID NO: 10) | |
| lpxE-R | GGGGGCTAGCTTAAATAATCTC | |
| | TCTATTTCTCATC | |
| | (SEQ ID NO: 11) | |
| pagL-F | GGGGGAATTCGGATCCGTGTAT | This study |
| | ATGAAGAG | |
| | (SEQ ID NO: 12) | |
| pagL-R | GGGGGCTAGCTCAGAAATTATA | |
| | ACTAATTG | |
| | (SEQ ID NO: 13) | |
| P_{ompC}-F | GAATTCTGTGGTAGCACAGA | This study |
| | (SEQ ID NO: 14) | |
| pSEC10M-F | TTTTGAATTCGCGGCCGCATTT | This study |
| | AAATGCTAGCAAAA | |
| | (SEQ ID NO: 15) | |
| pSEC10M-R | TTTTGCTAGCATTTAAATGCGG | |
| | CCGCGAATTCAAAA | |
| | (SEQ ID NO: 16) | |

TABLE 3
Plasmids used in this study

| Plasmid | Description | Genotype | Ref. |
|---|---|---|---|
| pSEC10 | Osmotic-inducible vector for gene | ori101, hok-sok, parA/B; | 14 |
| | expression under P_{ompC} | P_{ompC}-ClyA | |
| pSEC10M | Modified pSEC10 plasmid with P_{ompC} and | ori101, hok-sok, parA/B; | This |
| | clyA replaced by an MCS | P_{ompC}-clyA::MCS | study |
| pGRG36 | Arabinose-inducible Tn7 transposition | ori ts, oriT, araC, P_{BAD}- | 15 |
| | vector with Smal added to MCS site of | tnsABCD, mTn7::MCS | |
| | pGRG25 | | |

### Generation of Plasmid-Based BECC-Modified Shigella Strains

LPS modifying enzymes LpxE, PagL, and LpxE-PagL in tandem (termed “Dual”) were first cloned and expressed in pSEC10 under the osmotically controlled E. coli ompC promoter (P_{ompC}) (Stokes et al., Infect Immun, (2007), 75:1827-34). A codon-optimized form of lpxE from Francisella novicida was synthesized by GenScript and cloned into pUC57, yielding pUC57::lpxE. The 720 bp lpxE gene was amplified by PCR from pUC57::lpxE using Q5 polymerase (New England Biolabs) and primer set lpxE-F/lpxE-R, trimmed with restriction enzymes BamHI and NheI, and ligated into the BamHINheI site of pSEC10 resulting in the construct pSEC10::P_{ompC}-lpxE. The pagL gene was amplified by PCR from Salmonella minnesota (Genbank accession AE006468.2) using Q5 polymerase and primer set pagL-F/pagL-R. A 570 bp amplicon was trimmed with BamHINheI and ligated into the 6630 bp BamHI/NheI digested fragment of pSEC10 yielding pSEC10::P_{ompC}-pagL. Both lpxE and pagL, each preceded by a ribosomal binding site, were synthesized in tandem behind P_{ompC} and cloned into vector pUC57K by GenScript yielding pUC57K::P_{ompC}-Dual. The initial subcloning of pagL into pSEC10 included six additional bps (GTGTAT) that encoded an alternative start codon present in the S. minnesota sequence; this 6 bp sequence was not included in the Dual construct. The P_{ompC}-Dual gene cassette was then cloned into a modified version of pSEC10 (pSEC10M). Briefly, primer set pSEC10M-F/pSEC10M-R was self-annealed, trimmed with EcoRI/NheI, and ligated into a 5775 bp gel purified EcoRI-NheI digested pSEC10 and transformed into E. coli TOP10. The resulting pSEC10M had a multiple cloning site [EcoRI-NotI-SwaI-NheI] in place of the ompC promoter and clyA gene. The 1.9 kb SwaI P_{ompC}-Dual gene cassette isolated from pUC57K::P_{ompC}-Dual was ligated into the SwaI digested site of pSEC10M and transformed into E. coli TOP10 yielding construct pSEC10M::P_{ompC}-Dual. Plasmids pSEC10::P_{ompC}-lpxE, pSEC10::P_{ompC}-pagL, and pSEC10M::P_{ompC}-Dual were electroporated into the wild-type strains of S. sonnei and S. flexneri 2a and selected on TSA with neomycin. Successful transformants were used to inoculate a 2 mL overnight culture in TSB with neomycin, which received a final concentration of 15% glycerol, followed by storage at −80° C.

Generation of attTn7 Chromosomally Integrated BECC-Modified Shigella Strains

We mobilized P_{ompC}-lpxE and P_{ompC}-Dual into the Shigella chromosome attTn7 site using a site-specific insertion method utilizing the Tn7 recombination machinery on a temperature-sensitive plasmid pGRG36 (McKenzie et al., BMC Microbiology, (2006), 6:39). A 1225 bp amplicon of the P_{ompC}-lpxE gene cassette was generated using template pSEC10::P_{ompC}-lpxE, Q5 polymerase (New England Biolabs) and primer set P_{ompC}-F/lpxE-R. This was then blunt-ligated into the SmaI digested site of pGRG36, yielding pGRG36::PₒₘₚC-lpxE. A 1.9 kb P_{ompC}-Dual gene cassette flanked by SwaI restriction sites was isolated from pUC57K::P_{ompC}-Dual and ligated into the SwaI site of pGRG36 yielding pGRG36::P_{ompC}-Dual. The resulting pGRG36 constructs were transformed into E. coli S17-1 and introduced into wild-type and 2^{nd} generation vaccine strains of S. sonnei and S. flexneri 2a by conjugal mating. Briefly, 2 mL cultures were grown overnight. E. coli S17-1 plasmid transformants were grown in the presence of carbenicillin at 30° C. whereas Shigella cultures were grown without antibiotic at 37° C. Filter matings were performed by mixing 100 μL of Shigella with 50 μL of E. coli S17-1 plasmid transformants, concentrated by centrifugation (8k×g for 1 minute), resuspended in 200 μl TSB, spread onto a 0.45 μM nylon filter (MSI Magna nylon 66) placed on the center of a TSA plate, and incubated for 5 hours at 30° C. Bacteria on the nylon filter were then resuspended in 1 mL TSB and plated onto TSA containing 0.01% Congo red dye (Sigma-Aldrich), carbenicillin, and 1 mM MgCl₂ and incubated overnight at 30° C. Shigella conjugants that grew at 30° C. and were carbenicillin resistant were screened by PCR for the presence of lpxE and the Shigella invasion plasmid using GoTaq (Promega) using primer sets lpxE-F/lpxE-R and ospD3-F/ospD3-R or lpxE-F/lpxE-R and ipaB-F/ipaB-R for WT and vaccine strains, respectively. Bacteria were plated on TSA containing 0.1% arabinose and incubated at 42° C. overnight to promote Tn7 recombination and simultaneous curing of pGRG36. Isolates that were carbenicillin sensitive and Congo Red positive, were assayed by PCR for the presence of lpxE using primer set lpxE-F/lpxE-R and for the Shigella invasion plasmid using ospD3 primer set ospD3-F/ospD3-R or ipaB primer set ipaB-F/ipaB-R for WT and vaccine strains, respectively. Confirmed integrants were stored in 15% glycerol at −80° C. Bacterial genomes were sequenced at the Microbial Genome Sequencing Center (SeqCenter) and analyzed using the RAST software which confirmed the insertion of the gene cassettes into the attTn7 site (Aziz, R. K., et al., BMC Genomics, (2008), 9:75).

### MALDI-TOF MS and MS/MS Analysis of Lipid A

Functional screening of Shigella strains to confirm lipid A modification was performed using the Fast Lipid Analysis Technique (FLAT) coupled to MALDI-TOF MS analysis (Sorensen et al., Scientific Reports, (2020), 10). Briefly, a single colony was spotted on a MALDI plate and overlaid with 1 μL of citrate buffer (200 mM citric acid, 100 mM trisodium citrate, pH 3.5). The plate was incubated in a humidified, closed, glass chamber for 30 minutes at 110° C., cooled, washed with endotoxin-free water, and 1 μL of 10 mg/mL norharmane matrix (Sigma-Aldrich) dissolved in chloroform:methanol (2:1 v/v) was spotted onto the samples on the MALDI plate. MALDI-TOF MS analysis was performed using a Bruker Microflex LRF equipped with a 337 nm nitrogen laser. Spectra were acquired in the negative ion and reflectron mode. Analyses were conducted at <60% global intensity with 300 laser shots for each spectrum acquisition. Spectra were recorded in triplicate. Agilent ESI tune mix (Agilent) was used for mass calibration. FlexAnalysis software version 3.4 (Bruker) was used to process the mass spectra with smoothed and baseline corrections. Further structural lipid A characterization was conducted by tandem mass spectrometry (MS/MS) analysis using the FLATⁿ procedure (Yang et al., Anal Chem, (2022), 94:7460-7465). The FLAT process above was repeated, except the colony spotted onto an indium tin oxide (ITO) slide instead of a MALDI plate. MS/MS analysis was performed using a Bruker MALDI trapped ion mobility spectrometry Time-of-Flight (timsTOF) mass spectrometer equipped with a dual ESI/MALDI source with a SmartBeam 3D 10 KHz frequency tripled 355 nm Nd:YAG laser. The system was operated in “qTOF” mode (tims deactivated). Ion transfer tuning used the following parameters: Funnel 1 RF: 440.0 Vpp, Funnel 2 RF: 490.0 Vpp, Multipole RF 490.0 Vpp, is CID Energy: 0.0 eV, and Deflection Delta: −60.0 V. The quadrupole used the following values for MS mode: Ion Energy: 4.0 eV and Low Mass 700.00 m/z. Collision cell activation of ions used the following values for MS mode: Collision Energy: 9.0 eV and Collision RF: 3900.0 Vpp. The precursor ion was chosen by inputting targeted m/z values including two digits beyond the decimal point. Typical isolation width and collision energy were set to 4-6 m/z and 100-110 eV, respectively. Focus Pre-TOF used the following values: Transfer time 110.0 μs and Pre pulse storage 9.0 μs. Agilent ESI Tune Mix (Agilent) was used to perform calibration. MALDI parameters in qTOF mode were optimized to maximize intensity by tuning ion optics, laser intensity, and laser focus. All spectra were collected at a laser diameter of 104 μm with beam scan on using 800 laser shots per spot using either 70% or 80% laser power. MS/MS data were collected in negative ion mode. In all cases, a matrix of 10 mg/mL norharmane dissolved in chloroform:methanol (2:1 v/v) was used. mMass software version 5.5.0 was used to process the mass spectra with smoothed and baseline corrections. Identification of all fragment ions were determined based on ChemDraw Ultra version 10.0 (Niedermeyer et al., PLoS ONE, (2012), 7:e44913).

SDS-PAGE and Pro-Q Emerald 300 staining

LPS was isolated from small-scale (2 mL) cultures via hot aqueous-phenol extraction according to and separated by SDS-PAGE on NuPAGE 4-12% Bis-Tris precast gels (Invitrogen) at 150 V for 35 minutes (Davis et al., J Vis Exp, (2012), (63). The gel was fixed and stained according to the Pro-Q™ Emerald 300 Lipopolysaccharide Gel Stain Kit manufacturers protocol (Invitrogen) and exposed for 0.5 seconds using the Pro-Q Emerald 300 filter on a standard ChemiDoc imaging system (Bio-Rad).

### LPS Extraction and Purification

LPS was isolated using the double hot phenol method. Briefly, two liters of bacterial culture were harvested by centrifugation and resuspended in 90% phenol:endotoxin-free water (1:1 v/v) and incubated at 65° C. for 1 hour. After centrifugation, the aqueous phase was isolated from the two-phase solution (repeated three times total and pooled) and dialyzed for 36 hours against deionized water using pre-treated 1 kD MWCO RC tubing (Spectrumlabs.com), followed by flash freezing and lyophilization. The lyophilized product was resuspended in 20 mM Tris-HCl pH 8.4 supplemented with 2 mM MgCl₂ and digested using 500 units of Benzonase and 100 μg/mL Dnase I for 2 hours at 37° C. The pH was subsequently adjusted to 7.4 using 1 N HCl and the solution further digested with 100 μg/mL Proteinase K for 2 hours at 37° C. Water-saturated phenol was added, vortexed, and centrifuged (8,000×g), and the upper aqueous phase was collected, dialyzed, and lyophilized. Further isolation of LPS was performed by serial washes in chloroform:methanol (2:1 v/v) as described (Folch et al., J Biol Chem, (1957), 226: 497-509). The LPS was separated from contaminating lipoproteins as described by resuspension in 0.2% TEA (triethylamine) and 0.5% DOC (deoxycholate), followed by the addition of 37° C. water-saturated phenol and the upper aqueous phase collected (Hirschfeld et al., The Journal of Immunology, (2000), 165:618-622). Finally, the LPS product was precipitated by the addition of cold 100% ethanol and 30 mM sodium acetate followed by incubation for 18 hours at −20° C. The LPS precipitate was harvested by centrifugation (5,000×g, 20 minutes), washed in cold 100% ethanol, resuspended in endotoxin-free water (Quality Biological), and lyophilized.

### Kdo Assay for LPS Quantification

2-keto-3-deoxyoctonate (Kdo) standards ranging from 12-48 μg/mL in endotoxin-free water and 1 mg/mL LPS solution solutions were hydrolyzed in 0.018 N sulfuric acid (H₂SO₄) at 100° C. for 20 minutes, followed by the addition of 25 μL of 9.1 mg/mL periodic acid (H₅IO₆) in 0.125 N H₂SO₄ and incubation in the dark for 20 minutes. Samples then received 50 μL of 2.6% sodium arsenite (NaAsO2) in 0.5 N HCl followed by the addition of 250 μL of 0.3% thiobarbituric acid (TBA). Samples were heated at 100° C. for 10 minutes, quickly followed by the addition of 125 μL of dimethyl sulfoxide (DMSO), and the measurement of absorbance at 550 nm. The absolute quantification is based on the interpolation of the standard curve provided by the Kdo₂ quantity. Half of the Kdo₂ quantity, representing Kdo₁ (referred to as simply “Kdo” in this study), was utilized for normalization.

### Murine Acute Endotoxemia

LPS solutions of 45 μg/mL Kdo₂ (representative of 15 mg/kg if using dry weight instead) were prepared in sterile, endotoxin-free PBS (Quality Biology). LPS solutions were transferred to arbitrarily labeled tubes by a third-party observer to ensure blinding to group identifications and avoid bias in clinical score designations. Each mice received 100 μL of LPS solution using a slip tip 1 mL syringe attached to a 27-gauge ½ inch needle (Becton Dickinson) via the intraperitoneal route. Mice were monitored for 72 hours post-injection, receiving a clinical score/mouse based on appearance and mobility as described in Table 4. A clinical score of 5 required euthanization as a consequence of no movement, noticeable stress, and an inability to return upright if placed on their side.

TABLE 4
Clinical scores for mice during endotoxicity study

| Score | Description | Appearance | Mobility |
|---|---|---|---|
| 0 | Perfectly healthy | — | — |
| 1 | Smooth | Slightly ruffled coat | Active and scurrying, |
| | | | burrowing |
| 2 | Slightly ruffled | Ruffled coat | Active and scurrying, |
| | | | burrowing |
| 3 | Ruffled | Very ruffled coat | Walking, but no |
| | | | scurrying, mildly |
| | | | lethargic |
| 4 | Sick | Very ruffled coat, inset | Slow to no movement, |
| | | eyes | extremely lethargic |
| 5 | Very sick (euthanize) | Very ruffled coat, | No movement or |
| | | closed inset eyes | spastic movements, will |
| | | | not return upright if put |
| | | | on side, noticeable |
| | | | stress |
| 6 | Deceased | — | — |

### Cell Culture Media and Conditions

RPMI-1640 (Gibco) complemented with 25 mM HEPES, 2 mM glutamine, 10% FBS, and 1% penicillin-streptomycin, referred to as cRPMI, was filter sterilized through a 0.22 μM filter flask and used for the THP-1 NF-κB-SEAP reporter cell line (THP-1 Dual, Invitrogen). DMEM (Corning) complemented with 3.7 g/L sodium bicarbonate, 2 mM glutamine, 10% FBS, and 1% penicillin-streptomycin, referred to as cDMEM, was filter sterilized through a 0.22 μM filter flask and used for the HT29 cells (courtesy of Dr. Eileen Barry, UMB), mTLR4/hTLR4 HEK-Blue cells (Invitrogen), and RAW-Blue cells (Invitrogen). All cells were maintained at 37° C. with 5% CO₂.

### NF-κB Reporter Cell Line Stimulations

Sterile, cell culture-treated, 96-well flat bottom plates (Costar) were seeded with HEK-Blue, RAW-Blue, or THP-1 Dual reporter cells at 6×10⁴ cells/well. THP-1 cells received 100 ng/mL of vitamin D₃ (Sigma) prior to seeding in wells to enable cell differentiation. Cells were incubated for 18 hours at 37° C. with 5% CO₂, except the THP-1 cells, which were incubated for 72 hours to enable differentiation into monocyte-derived macrophages. Five 10-fold dilutions of Kdo standardized LPS ranging from 10² pg/mL to 10⁻² pg/mL was used to stimulate NF-κB production in cells. Cells were incubated at 37° C. with 5% CO₂ for 18 hours. Detection of NF-κB was quantified using the Quanti-Blue (QB) reagent prepared according to the manufacturer's protocols (Invitrogen). The percent of relative NF-κB activation was normalized to the maximum OD 630 nm measured. Points were plotted as the mean±standard deviation of the relative NF-κB activation at each concentration using GraphPad Prism version 9 and fitted using a nonlinear regression of the log(agonist) versus response (three parameters).

### Stimulation of Primary Peripheral Blood Monocytes

Human peripheral blood was collected from healthy adult study participants 18-40 years of age per a Boston Children's IRB-approved protocol (protocol number X07-05-0223). All participants signed an informed consent form prior to enrollment. Heparinized whole blood was centrifuged (500×g, 10 minutes) prior to removal of the upper layer of platelet-rich plasma. The plasma was centrifuged (3,000×g, 10 minutes) and platelet-poor plasma (PPP) was collected and stored on ice. The remaining blood was reconstituted to its original volume with heparinized Dulbecco's PBS and layered on Ficoll-Paque gradients (Cytiva) in Accuspin tubes (Sigma-Aldrich). PBMCs were collected after centrifugation, washed twice with PBS, and seeded at 2×10⁵ cells/well in 96-well U-bottom plates (Corning) in RPMI-1640 media (Gibco) supplemented with 10% autologous PPP, 100 IU/mL penicillin, 100 g/mL streptomycin, and 2 mM L-glutamine. PBMCs were incubated with 1 μg/mL Kdo standardized LPS for 24 hours at 37° C. with 5% CO₂. The supernatants were recovered after centrifugation (500×g, 5 minutes) and analyzed for TNF-α quantification.

For multiplex analysis, frozen PBMCs from 4 independent human donors were instead obtained from AllCells, snap-thawed, washed twice with warm cRPMI, and seeded at 5×10⁵ cells/well in 96-well, sterile, uncoated U-bottom plates (Costar). PBMCs were incubated with 1 μg/mL Kdo standardized LPS for 48 hours at 37° C. with 5% CO₂. The supernatants were recovered after centrifugation (400×g, 5 minutes) and stored at −20° C. until analyzed by MSD multiplex.

### Invasion Assays

HT29 cells were seeded at a density of 5×10⁵ cells/well in 24-well flat bottom sterile plates (Corning) and incubated at 37° C. for 18 hours with 5% CO₂. Shigella cultures from overnight growth on TSA containing 0.01% Congo Red were used to generate a resuspension in sterile PBS pH 7.4. Inoculums of 5×10⁶ CFU in serum free media were used to infect, giving an MOI of 10. Enumeration of the inoculation was confirmed in duplicate by plate counts on TSA. Bacterial inoculums were added to PBS-washed cell monolayers and the plates were centrifuged (3,000×g, 5 minutes) to start the infection. After incubation for 90 minutes at 37° C. with 5% CO₂, cell monolayers were twice washed with sterile PBS, followed by the addition of cDMEM containing 50 g/mL gentamycin (Sigma-Aldrich) and incubation for 2.5 hours. Supernatants were isolated and processed for human CXCL8 secretion by cytokine ELISA. The cell monolayer was twice washed with sterile PBS and lysed with 1% Triton X-100 (Sigma-Aldrich) in sterile PBS for 10 minutes at room temperature. Serial dilutions were plated in duplicate on TSA and incubated overnight at 37° C. The percentage of invasion was determined as the CFU/mL recovered normalized to the CFU/mL inoculated.

### Cytokine ELISA

Cytokine analysis of host cell culture supernatants was performed using DuoSet ELISA kits (R&D Systems) according to the manufacturer's protocol. Briefly, plates were coated overnight at 4° C. by adding 100 μL/well of 2 μg/mL capture antibody in ELISA coating buffer, washed three times PBS+0.02% Tween-20 (PBST) and blocked with 300 μL/well 1% BSA in PBS for 1 hour at room temperature followed by three washes with PBST. Cell culture supernatants were diluted to reach a signal within the dynamic range. Bound cytokines were labeled by adding biotin-conjugated antibodies in block buffer (100 μL/well of 2 μg/mL) and incubated at room temperature for 2 hours. Plates were washed with PBST and incubated for 20 minutes with secondary antibody streptavidin-HRP, followed by the addition of a color substrate. The plates were read at both 450 nm and 562 nm and the difference taken as the final reading. The amount of cytokine is reported as picograms per mL of cell culture supernatant.

Murine Vaccination with Live-Attenuated Shigella

Shigella vaccine strains were grown at 37° C. overnight on TSA containing 0.01% Congo Red. For intranasal vaccination, inoculums of 3.33×10⁸ CFU/mL and 3.33×10⁷ CFU/mL of Shigella were prepared in sterile PBS and kept at room temperature. Mice were anesthetized using a Matrx VIP 3000 vaporizer (Midmark Animal Health) with isoflurane (Fluriso, VetOne): oxygen (Airgas, OX USPEAWBDS) mixture (1:1 mixing) for 1-2 minutes and 15 μL of the vaccine inoculum was delivered to each nare (30 μL in total) using a pipette. For oral gastric vaccination, inoculums of 1×10⁸ CFU/mL and 1×10⁷ CFU/mL of Shigella were prepared in sterile PBS and kept at room temperature. Inoculums of 100 μL were delivered by intra-gastric gavage using a 2-inch-long plastic feeding needle (VWR) connected to a 1 mL syringe (Becton Dickinson). For both vaccination methods, mice were monitored for adverse reactions post-immunization. Enumeration of the vaccine inoculums was determined by plate counts on TSA.

Murine Vaccination with Purified LPS

Purified LPS from wild-type S. sonnei Moseley was obtained as described above (see LPS extraction and purification). For internasal vaccination, solutions containing purified LPS at 1 mg/mL and 0.66 mg/mL, dissolved in sterile PBS, were delivered intranasally as described above. For intramuscular vaccination, solutions containing purified LPS at 0.6 mg/mL and 0.4 mg/mL, dissolved in sterile PBS, were prepared and stored at room temperature. Mice were immobilized using a restrainer, and 50 μL of the solution was injected using a 1 mL syringe (Becton Dickinson) into the caudal muscle after disinfecting the area with 70% ethanol. For both vaccination methods, mice were monitored for adverse reactions post-immunization.

### Sera Collection

Mice were bled via the lateral saphenous vein using petroleum jelly and 27-gauge needles. Blood was collected in a microvette 200 Z-gel tubes (Sarstedt), and the sera were isolated by centrifugation (10,000×g, 3 minutes) and stored in sealed uncoated 96-well flat bottom plates (Thermo Fischer) at −20° C.

### Enzyme-Linked Immunosorbent Assay (ELISA)

Coating antigens used in ELISAs included purified LPS from wild-type S. sonnei Moseley or S. flexneri 2a 2457T. Nunc MaxiSorp plates (ThermoFischer) were coated with 5 μg/mL serotype-specific LPS in 100 mM carbonate coating buffer pH 9.6 (sodium bicarbonate/carbonate) and incubated for 3 hours at 37° C. Plates were washed with PBS containing 0.05% Tween-20 (Sigma) (PBST) and blocked with 10% non-fat dry milk powder (Quality Biological) in PBST overnight at 4° C. Sera was diluted in 5-fold increments starting with a 1:50 dilution in PBST, added to the LPS-coated plates, and incubated at 37° C. for 2 hours. Plates were washed with PBST. Incubation for 1 hour with secondary HRP-conjugated antibodies, goat anti-mouse IgG, IgG1, IgG2a (Southern Biotech) or goat anti-mouse IgA (Invitrogen) was followed by a 15 minute room temperature incubation with 3,3′,5,5′-tetramethylbenzidine (TMB) substrate (BD biosciences) prepared according to manufacturer's protocol. KPL TMB stop solution (Sera Care) containing 1% HCl was added to each well, and the absorbance read at 450 nm. The endpoint titer was determined as the absorbance reading that was equal to the reciprocal dilution required for the signal to match the average blank (PBST alone, no sera). Samples were run in duplicate. Sera from days 28, 42, and 56 of the vaccine study required dilutions of 1:1250 for IgG and IgG1 titers and 1:250 for IgG2a to reach specific endpoints.

### Multiplex Cytokine Analysis

MSD (Meso Scale Development) V-PLEX human proinflammatory panel 1 (10-plex) was used for the analysis of the human orthologs of IFNγ, IL-1β, IL-2, IL-4, IL-6, CXCL8, IL-10, IL-12p70, IL-13 and TNF-α from 25 μL of 6-fold dilutions of the supernatant from the stimulation of human PBMCs; however only data from IL-1β, IL-6, IL-10, and TNF-α were used for this study. Samples and calibrators were incubated at room temperature, shaking (500 RPM), for 2 hours. Plates were washed with PBST and MSD detection antibody solution, prepared according to the manufacturer's protocol, added and incubated shaking (500 RPM) at room temperature for 2 hours. Plates were washed with 150 μL/well PBST followed by the addition of 150 μL/well of read buffer T. Plates were immediately read on an MSD SQ 120/120MM instrument. Cytokine concentration was determined by interpolation from a standard curve generated using the provided calibrators. Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

We claim:

1. A genetically modified Shigella microorganism with reduced endotoxicity, wherein the Shigella is genetically modified to express a lipid A 1-phosphatase.

2. The genetically modified Shigella microorganism of claim 1, wherein the lipid A 1-phosphatase is LpxE.

3. The genetically modified Shigella microorganism of claim 2, wherein the LpxE is from Francisella novicida.

4. The genetically modified Shigella microorganism of claim 1, wherein the lipid A 1-phosphatase is codon optimized for expression in Shigella.

5. The genetically modified Shigella microorganism of claim 2, wherein the LpxE has an animo acid sequence comprising SEQ ID NO:1.

6. The genetically modified Shigella microorganism of claim 1, wherein the Shigella is genetically modified to express a 3-O deacylase, wherein the 3-O deacylase is PagL.

7. The genetically modified Shigella microorganism of claim 6, wherein the PagL is from Salmonella enterica.

8. The genetically modified Shigella microorganism of claim 7, wherein the PagL has an animo acid sequence comprising SEQ ID NO:3.

9. The genetically modified Shigella microorganism of claim 1, wherein the lipid A 1-phosphatase is chromosomally integrated or expressed from a plasmid.

10. The genetically modified Shigella microorganism of claim 1, wherein the lipid A 1-phosphatase is regulated by an inducible promoter.

11. The genetically modified Shigella microorganism claim 10, wherein the inducible promoter is an osmotically controlled E. coli ompC promoter (P_{ompC}).

12. A genetically modified Gram-negative microorganism with reduced endotoxicity, wherein the microorganism is genetically modified to express a lipid A 1-phosphatase.

13. The genetically modified Gram-negative microorganism of claim 12, wherein the Gram-negative microorganism is selected from Shigella, Escherichia coli, Pseudomonas aeruginosa, Salmonella, Vibrio and Yersinia species.

14. The genetically modified Gram-negative microorganism of claim 12, wherein the lipid A 1-phosphatase is LpxE.

15. The genetically modified Gram-negative microorganism of claim 14, wherein the LpxE is from Francisella novicida.

16. The genetically modified Gram-negative microorganism of claim 12, wherein the lipid A 1-phosphatase is codon optimized for expression in the Gram-negative microorganism.

17. The genetically modified Gram-negative microorganism of claim 14, wherein the LpxE has an animo acid sequence comprising SEQ ID NO:1.

18. The genetically modified Gram-negative microorganism of claim 14, wherein the LpxE is encoded by a nucleic acid sequence comprising SEQ ID NO:2.

19. The genetically modified Gram-negative microorganism of claim 12, wherein the Gram-negative microorganism is genetically modified to express a 3-O deacylase.

20. The genetically modified Gram-negative microorganism of claim 19, wherein the 3-O deacylase is PagL.


SEQUENCE LISTING (16 sequences)

SEQ ID NO: 1 - protein, 239 aa, synthetic construct
MLKQTLQTNFQGFKDIFKKPKLHNHKLPRYLQLKYTFIPLLILVIFAYYNLDTPVENYIKHSMPNIVGVIFGKITDVGKAEYILIICGVIVLARLFTDSQKLSANTRAMFDKVSAYAGFILATVAISGILGQILKMIIGRARPKFFLEYGSHYFQHFHAPGYDFASMPSGHSITVGAMFIAFFYIFPKLRYFWYLLIVVFAGSRIMVGSHYPSDVIFGVAFGCYCTAYIYYWMRNREII

SEQ ID NO: 2 - DNA, 720 bp, synthetic construct
  source 1..720: mol_type = other DNA
atgctcaaacagaccttacaaaccaactttcaaggttttaaagatatttttaaaaaaccgaaactgcacaatcataaattgcccagatacctgcagttgaaatatacgtttattccgttattaattttggtaatttttgcatactataacttagataccccggttgagaactatatcaagcattctatgccgaatattgttggtgtaatttttggtaaaattacggatgttggtaaggccgagtatattttgattatttgcggtgtgattgtgttagcgcgtttatttaccgatagccaaaaattatctgctaatacgagagctatgtttgacaaggtgtcggcatatgcgggttttatcttagcaacggtagctattagcggtattttgggacaaattctcaagatgattattggtagagcgcgtcctaagtttttcttggaatatggttcgcattatttccaacattttcatgcacccggatatgattttgcaagcatgccgtccgggcattccatcaccgttggagcaatgtttattgcatttttttatattttccctaagctgagatatttttggtatttgctgattgtggtatttgctgggagcagaattatggttggttcacattatccgagcgatgtaatttttggcgttgcttttggttgctactgtaccgcatatatctactattggatgagaaatagagagattatttaa

SEQ ID NO: 3 - protein, 185 aa, synthetic construct
MKRIFIYLLLPCAFACSANDNVFFGKGNKHQISFAAGESIRRGGVEHLYTAFLTYSEPSDFFFLQARNNLELGGFKAKGSDDCSKHSGSVPCNKYNQGVLGISKDVALVHFAGIYTGIGLGAYIKSKSRDDMRVNSAFTFGEKAFLGWNFGAFSTEAYIRHFSNGSLTDKNSGHNFVGASISYNF

SEQ ID NO: 4 - DNA, 558 bp, synthetic construct
  source 1..558: mol_type = other DNA
atgaagagaatatttatatatctattattaccttgtgcattcgcatgttctgctaatgataatgttttttttggcaagggcaacaagcatcagatctcttttgctgcgggagaaagtataagaagaggaggggttgagcacttatatacggcttttctgacatacagtgaacccagcgattttttctttttacaggcaagaaataatctggagttaggaggatttaaggctaagggtagcgatgattgcagtaaacattctggcagcgttccctgtaataaatataaccagggcgtattgggtatctcgaaggatgtggcgctggttcatttcgctggtatctataccggtattggtctgggggcttatataaaatctaagtcgcgagatgatatgcgtgtcaattctgcatttacctttggagaaaaagcgtttcttggctggaactttggggctttttctacagaagcttatatccggcatttctcgaatggatcacttacggataaaaattcagggcataattttgtaggtgcttcaattagttataatttctga

SEQ ID NO: 5 - DNA, 1215 bp, synthetic construct
  source 1..1215: mol_type = other DNA
gaattctgtggtagcacagaataatgaaaagtgtgtaaagaagggtaaaaaaaaccgaatgcgaggcatccggttgaaataggggtaaacagacattcagaaatgaatgacggtaataaataaagttaatgatgatagcgggagttattctagttgcgagtgaaggttttgttttgacattcagtgctgtcaaatacttaagaataagttattgattttaaccttgaattattattgcttgatgttaggtgcttatttcgccattccgcaataatcttaaaaagttcccttgcatttacattttgaaacatctatagcgataaatgaaacatcttaaaagttttagtatcatattcgtgttggattattctgcatttttggggagaatggacttgccgactgattaatgagggttaatcagtatgcagtggcataaaaaagcaaataaaggcatataacagatcgatcttaaacatccacaggaggatgggatccatgctcaaacagaccttacaaaccaactttcaaggttttaaagatatttttaaaaaaccgaaactgcacaatcataaattgcccagatacctgcagttgaaatatacgtttattccgttattaattttggtaatttttgcatactataacttagataccccggttgagaactatatcaagcattctatgccgaatattgttggtgtaatttttggtaaaattacggatgttggtaaggccgagtatattttgattatttgcggtgtgattgtgttagcgcgtttatttaccgatagccaaaaattatctgctaatacgagagctatgtttgacaaggtgtcggcatatgcgggttttatcttagcaacggtagctattagcggtattttgggacaaattctcaagatgattattggtagagcgcgtcctaagtttttcttggaatatggttcgcattatttccaacattttcatgcacccggatatgattttgcaagcatgccgtccgggcattccatcaccgttggagcaatgtttattgcatttttttatattttccctaagctgagatatttttggtatttgctgattgtggtatttgctgggagcagaattatggttggttcacattatccgagcgatgtaatttttggcgttgcttttggttgctactgtaccgcatatatctactattggatgagaaatagagagattatttaa

SEQ ID NO: 6 - DNA, 22 bp, synthetic construct
  source 1..22: mol_type = other DNA
tacacgtccattatgcaaggct

SEQ ID NO: 7 - DNA, 25 bp, synthetic construct
  source 1..25: mol_type = other DNA
tgccatcagtaaatttaatcccatc

SEQ ID NO: 8 - DNA, 20 bp, synthetic construct
  source 1..20: mol_type = other DNA
cacagcatctgctgaacagc

SEQ ID NO: 9 - DNA, 20 bp, synthetic construct
  source 1..20: mol_type = other DNA
cagcagaagcgacacttcct

SEQ ID NO: 10 - DNA, 21 bp, synthetic construct
  source 1..21: mol_type = other DNA
ggatccatgctcaaacagacc

SEQ ID NO: 11 - DNA, 35 bp, synthetic construct
  source 1..35: mol_type = other DNA
gggggctagcttaaataatctctctatttctcatc

SEQ ID NO: 12 - DNA, 30 bp, synthetic construct
  source 1..30: mol_type = other DNA
gggggaattcggatccgtgtatatgaagag

SEQ ID NO: 13 - DNA, 30 bp, synthetic construct
  source 1..30: mol_type = other DNA
gggggctagctcagaaattataactaattg

SEQ ID NO: 14 - DNA, 20 bp, synthetic construct
  source 1..20: mol_type = other DNA
gaattctgtggtagcacaga

SEQ ID NO: 15 - DNA, 36 bp, synthetic construct
  source 1..36: mol_type = other DNA
ttttgaattcgcggccgcatttaaatgctagcaaaa

SEQ ID NO: 16 - DNA, 36 bp, synthetic construct
  source 1..36: mol_type = other DNA
ttttgctagcatttaaatgcggccgcgaattcaaaa